(19) United States
(12) Patent Application Publication
(10) Pub. No.: US 2024/0410831 A1
(43) Pub. Date: Dec. 12, 2024

(54) Multi-Mode Zinc-Selective Emission-Ratiometric Fluorescent Probes for Biological Application

(71) Applicant: Georgia Tech Research Corporation, Atlanta, GA (US)

(72) Inventors: Christoph J. Fahrni, Atlanta, GA (US); Jiyao Yu, Atlanta, GA (US)

(21) Appl. No.: 18/736,425

(22) Filed: Jun. 6, 2024

Related U.S. Application Data
(60) Provisional application No. 63/506,443, filed on Jun. 6, 2023.

(51) Int. Cl. G01N 21/64; G01N 33/20

(52) U.S. Cl. CPC G01N 21/6458; G01N 33/20

(57) ABSTRACT

Compounds, compositions, and methods for detecting zinc in a sample. The compounds are related to zinc-sensitive fluorescence probes. The compounds provide an emission-ratiometric fluorescence response upon binding of an analyte. The compounds can be used for two-photon excitation microscopy or conventional fluorescence microscopy. The compounds can be configured to shift the emission maximum by about 10 nm to about 60 nm upon formation of a complex with Zn(II).

### CROSS-REFERENCE TO RELATED APPLICATIONS

This application claims the priority benefit of U.S. Provisional Patent Application Ser. No. 63/506,443, filed Jun. 6, 2023, which is hereby incorporated by reference in its entirety.

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### SEQUENCE LISTING

Not Applicable

### STATEMENT REGARDING PRIOR DISCLOSURES BY THE INVENTOR OR A JOINT INVENTOR

Not Applicable

### BACKGROUND OF THE DISCLOSURE

### 1. Field of the Invention

The present invention relates generally to zinc-responsive fluorescent probes, compositions, and methods utilizing the same and in particular to multi-modal emission-ratiometric fluorescent probes which can be utilized both in non-linear two-photon excitation microscopy (TPEM) and traditional linear optical fluorescence detection methods such as laser confocal fluorescence microscopy for the selective detection of labile Zn(II) pools in biological samples such as live cells, tissues, and whole organisms.

### 2. Description of Related Art

Zinc (Zn) is an essential trace nutrient required for the vitality of all living organisms. It plays a central role in major cellular processes, including gene regulation, protein synthesis, and metabolic pathways. Cell development and proliferation critically depend on the presence of zinc ions. For example, the requirements for zinc are increased during pregnancy and lactation and hence critical to growth and development of the fetus and neonate. Zinc also plays an important role in bone metabolism and is essential for the normal development of the skeleton in humans and animals. The availability of zinc generally affects processes that involve rapid proliferation, such as wound healing, angiogenesis, and tumor growth. At the cellular level, zinc deprivation inhibits proliferation via cell cycle arrest at the late G1 phase as well as the G2/M transition, and blocks progression past telophase I of the meiotic cell cycle. Conversely, zinc supplementation promotes proliferation of healthy mouse cells as well as tumor cells.

Despite the importance of zinc in biology, mechanisms governing zinc regulation and redistribution within cells, tissues, or whole organisms remain largely unexplored. Progress has been hampered in part due to the challenges associated with quantifying the distribution of zinc in situ. The total concentration of Zn in eukaryotic cells lies in the high micromolar to low millimolar range, which corresponds to 108-109 atoms per cell. Regulated through an intricate network of Zn-selective membrane transporters, cells are capable of maintaining intracellular Zn levels approximately two orders of magnitude higher compared to the extracellular environment. While the majority of intracellular Zn is bound to proteins, either as catalytic or structural component, cells also maintain a labile subpool that can readily exchange with exogenous chelators. According to measurements with a range of synthetic and genetically encoded fluorescent probes, this labile cytosolic Zn pool is buffered at picomolar to low nanomolar concentrations.

Several modern microanalytical techniques, notably secondary ion mass spectrometry, nuclear microprobes, and synchrotron x-ray fluorescence microscopy (microXRF), offer much improved detection limits compared to traditional methods such as inductively coupled plasma mass spectrometry and are also capable of quantifying trace metal within single cells with submicron spatial resolution. However, these methods can report only on total trace metal levels and are not suitable for analyzing dynamic changes in live cells.

The high-intensity laser excitation of confocal microscopes may elicit phototoxicity and photobleaching, both of which can have deleterious effects on delicate biological specimens such as live cells. In contrast, two-photon excitation microscopy (TPEM) is based on the simultaneous absorption of two low-energy near-infrared photons, resulting in reduced photo toxicity, increased depth penetration, and negligible background fluorescence. For this reason, TPEM represents the superior method for prolonged in vivo imaging studies.

There is a need for multi-mode emission-ratiometric fluorescent probes exhibiting visible light excitation that can be utilized by two-photon excitation microscopy and other traditional linear optical fluorescence detection techniques, including laser confocal fluorescence microscopy, light sheet microscopy, super resolution microscopy, or flow cytometry. It is to such a composition and method that embodiments of the invention are directed.

### SUMMARY OF THE INVENTION

Briefly described, according to exemplary embodiments of the present invention, compounds and methods. An embodiment of the present disclosure can be a compound according to Formula I:

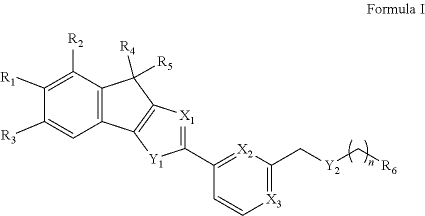

In the example embodiment, n may be 1, 2, or 3. In the example embodiment, Y₁ may be selected from —O—, —S—, —N(R⁴)—, and —C(R⁴)(R₅)—. In the example embodiment, Y₂ may be selected from —NR₇, —S—, and —O—; wherein R₇ is selected from C₁₋₃-alkyl optionally substituted with OR₈, —COOH, —COOR₈, —NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl. In the example embodiment, X₁, X₂, and X₃ are independently selected from —CH— and —N—. In the example embodiment, R₁ may be selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, substituted or unsubstituted heteroaryl, —OCH₃, —OCH₂COOR₉, —OCH₂CH₂COOR₉, —OCH₂CH₂CH₂SO₃R₉, —NHR₉, and —N(R₉)₂.

In the example embodiment, R₂ may be selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl. In some embodiments, R₁ and R₂ may join together to form a donor selected from substituted or unsubstituted heterocycloalkyl, substituted or unsubstituted heterocycloalkenyl, and substituted or unsubstituted heteroaryl.

In the example embodiment, R₃ may be selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl. In some embodiments, R₁ and R₃ may join together to form a donor selected from substituted or unsubstituted heterocycloalkyl, substituted or unsubstituted heterocycloalkenyl, and substituted or unsubstituted heteroaryl. In the example embodiment, R₄ and R₅ may be each independently selected from hydrogen, substituted or unsubstituted alkyl, alkenyl, alkynyl, cycloalkyl, cycloalkenyl, aryl, heteroakyl, heteroalkenyl, heteroalkynyl, heterocycloalkyl, heterocycloalkenyl, and heteroaryl. In various embodiments, R₄ and R₅ may join together to form a moiety selected from cycloalkyl, cycloalkenyl, aryl, heterocycloalkyl, heterocycloalkenyl, and heteroaryl.

In the example embodiment, R₆ may be selected from C₁₋₃-alkyl optionally substituted with —OR₈, —COOH, —COOR₈, —NHCOR₈, —NH₂, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl. In the example embodiment, R₈ may be selected from hydrogen, substituted or unsubstituted alkyl, substituted or unsubstituted aryl, substituted or unsubstituted heterocycloalkyl, substituted or unsubstituted heteroalkyl, and substituted or unsubstituted heteroaryl. In the example embodiment, R₉ may be independently selected from hydrogen, —COR₈, —SO₂—R₈, substituted or unsubstituted alkyl, substituted or unsubstituted aryl, substituted or unsubstituted heteroalkyl, substituted or unsubstituted heteroalkenyl, substituted or unsubstituted heteroalkynyl, and substituted or unsubstituted heteroaryl.

In various embodiments, Formula I may be selected from one or more derivative compounds. The one or more derivative compounds may comprise a modified electron donor moiety (e.g., Component A), a modified bridging unit (e.g., Component B), a modified chelator moiety (e.g., Component C), or a combination thereof. In various embodiments, R₁₂, R₁₃, R₁₄, R₁₅, and R₁₆ may be each independently selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl.

In various embodiments, R₁₇ and R₁₉ may be each independently selected from C₁₋₃-alkyl optionally substituted with —OR₈, —COOH, —COOR₈, —NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl. In various embodiments, R₁₈ may be selected from a group consisting of hydrogen, substituted or unsubstituted heterocycloalkyl, substituted or unsubstituted heteroalkyl, and substituted or unsubstituted heteroaryl. In one or more embodiments, a compound may comprise a conformationally locked core configured to be substituted with an electron donor moiety, wherein the substituted electron donor moiety is configured to fuse to a pyridine ring, wherein the pyridine ring is configured to act as an electron acceptor and metal ion binding moiety.

In various embodiments, a compound may allow for the modification of at least one of the following: electron donor moiety, bridging unit, chelator moiety, or a combination thereof. In various embodiments, the modification of a compound may be configured to tune the spectral characteristics, attach functional groups for bioconjugation with proteins, immobilization on solid support, tailor an affinity for a specific application, or a combination thereof. In various embodiments, a compound may be configured to exhibit a maximum absorption between about 400 nm and 500 nm for visible light or laser excitation. In various embodiments, a compound may be configured to exhibit a maximum two-photon absorption cross section between about 750 nm and 950 nm for two-photon excitation.

In another embodiment of the present disclosure, an embodiment of the present disclosure can be a compound according to Formula II:

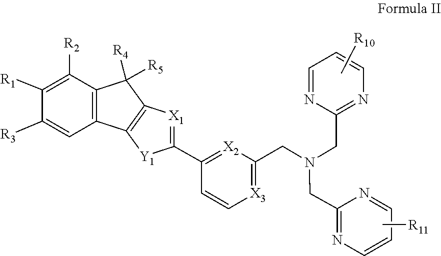

In the example embodiment, Y₁ may be selected from —O—, —S—, —N(R₄)—, and —C(R₄)(R₅)—. In the example embodiment, Y₂ may be selected from —NR₇, —S—, and —O—; wherein R₇ is selected from C₁₋₃-alkyl optionally substituted with —OR₈, —COOH, —COOR₈, —NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl. In the example embodiment, X₁, X₂, and X₃ are independently selected from —CH— and —N—. In the example embodiment, R₁ may be selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, substituted or unsubstituted heteroaryl, —OCH₃, —OCH₂COOR₉, —OCH₂CH₂COOR₉, —OCH₂CH₂CH₂SO₃R₉, —NHR₉, and —N(R₉)₂.

In the example embodiment, R₂ may be selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl. In some embodiments, R₁ and R₂ may join together to form a donor selected from substituted or unsubstituted heterocycloalkyl, substituted or unsubstituted heterocycloalkenyl, and substituted or unsubstituted heteroaryl.

In the example embodiment, R₃ may be selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl. In some embodiments, R₁ and R₃ may join together to form a donor selected from substituted or unsubstituted heterocycloalkyl, substituted or unsubstituted heterocycloalkenyl, and substituted or unsubstituted heteroaryl. In the example embodiment, R₄ and R₅ may be each independently selected from hydrogen, substituted or unsubstituted heterocycloalkyl, substituted or unsubstituted heteroalkyl, and substituted or unsubstituted heteroaryl.

In the example embodiment, R₈ may be selected from hydrogen, substituted or unsubstituted alkyl, substituted or unsubstituted aryl, substituted or unsubstituted heterocycloalkyl, substituted or unsubstituted heteroalkyl, and substituted or unsubstituted heteroaryl. In the example embodiment, R₉ may be independently selected from hydrogen, —COR₈, —SO₂—R₈, substituted or unsubstituted alkyl, substituted or unsubstituted aryl, substituted or unsubstituted heteroalkyl, substituted or unsubstituted heteroalkenyl, substituted or unsubstituted heteroalkynyl, and substituted or unsubstituted heteroaryl. In the example embodiment, R₁₀ and R₁₁ may be each independently selected from —H, —CH₃, —OR₈, —CF₃, —F, —Cl, —CN, —COOR₈, and —SO₃R⁸.

In various embodiments, the compound may be configured to exhibit a maximum absorption between about 380 nm and 500 nm for visible light or laser excitation. In various embodiments, the compound may be configured to exhibit a maximum two-photon absorption cross section between about 720 nm and 950 nm for two-photon excitation.

In one or more embodiments, a compound according to Formula I, Formula II, and/or a derivative thereof may be configured to be utilized as an emission-ratiometric fluorescent probe for the detection of Zn(II) in at least one of the following processes: visible-light fluorescence microscopy, confocal laser fluorescence microscopy, light sheet fluorescence microscopy, two-photon excitation microscopy, fluorescence lifetime imaging microscopy (FLIM), flow cytometry, microplate fluorescence detection analysis, steady-state fluorescence spectroscopy, lifetime fluorescence spectroscopy, or a combination thereof.

In various embodiments, a fluorescent complex may comprise a compound according to Formula I, Formula II, and/or a derivative thereof and zinc(II) comprising Formula I-Zn(II) and/or Formula II-Zn(II) in a 1:1 stoichiometric ratio. In various embodiments, the fluorescent compound may further comprise a ligand. In various embodiments, a maximum absorption wavelength of the Formula I-Zn(II) complex may be from about 10 nm to about 60 nm greater than the maximum absorption wavelength of the compound of Formula I. In various embodiments, a maximum absorption wavelength of the Formula II-Zn(II) complex may be from about 10 nm to about 60 nm greater than the maximum absorption wavelength of the compound of Formula II.

In yet another embodiments of the present disclosure, a method for detecting zinc in a sample is provided. The method may include treating a sample with a compound according to Formula I, Formula II, and/or a derivative thereof. The method may also include detecting an integrated fluorescence emission intensity over two different wavelength ranges. The first wavelength range may be dominated by fluorescence emission of the compound and the second wavelength range may be dominated by fluorescence emission of the zinc(II) complex of the compound. The method may further include comparing a first integrated emission intensity wavelength range to a second integrated emission intensity wavelength range.

In various embodiments, a compound according to Formula I, Formula II, and/or a derivative thereof may be configured to exhibit a fluorescence emission maximum between about 420 to 650 nm. The emission maximum of the Zn(II) complex of a compound may be from about 10 nm to about 60 nm greater than the emission maximum the compound alone. In one or more embodiment, the method may be configured to utilize two-photon excitation, such that the compound may shift the emission maximum by about 10 nm to about 60 nm upon formation of a complex with Zn(II). In various embodiments, the method may further comprise detecting Zn(II) using ratiometric fluorescence analysis of the compound emission over two different wavelength ranges. Ratiometric fluorescence analysis may compare the integrated emission intensity over first wavelength to the integrated emission intensity over the second wavelength range. In one or more embodiment, the method may further comprise determining a fractional saturation of the compound with Zn(II) by comparing the first integrated emission intensity wavelength range to the second integrated emission intensity wavelength range. The fractional saturation of the compound with Zn(II) may be configured to determine the concentration and/or activity of Zn(II) in the treated sample.

These and other aspects, features, and benefits of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

Implementations, features, and aspects of the disclosed technology are described in detail herein and are considered a part of the claimed disclosed technology. Other implementations, features, and aspects can be understood with reference to the following detailed description, accompanying drawings, and claims. Wherever possible, the same reference numbers are used throughout the drawings to refer to the same or like members of an embodiment. Reference will now be made to the accompanying figures and flow diagrams, which are not necessarily drawn to scale.

FIG. 1A illustrates an exemplary schematic of a compound configured for visible-light excitation with an emission-ratiometric response upon complexation with Zn(II) in accordance with various embodiments of the present disclosure.

FIG. 1B illustrates an exemplary schematic of a compound configured for visible-light excitation with an emission-ratiometric response upon complexation with Zn(II) in accordance with various embodiments of the present disclosure.

FIG. 2A provides a schematic illustration of a Zn(II)-responsive donor-acceptor substituted fluorophore architectures with a thiazole pi-bridge in accordance with various embodiments of the present disclosure.

FIG. 2B provides photophysical properties of Zn(II)-responsive donor-acceptor substituted fluorophore architectures in accordance with various embodiments of the present disclosure.

FIGS. 3A-3D illustrate exemplary variations of the donor moiety of an exemplary multi-mode Zn(II)-selective emission-ratiometric probe in accordance with various embodiments of the present disclosure.

FIGS. 3E-3I illustrate exemplary affinity tunings of an exemplary multi-mode Zn(II)-selective emission-ratiometic probe in accordance with various embodiments of the present disclosure.

FIGS. 3J-3L illustrate exemplary bridge modifications of an exemplary multi-mode Zn(II)-selective emission-ratiometic probe in accordance with various embodiments of the present disclosure.

FIGS. 4A-4H illustrate a portion of an exemplary synthetic scheme for accessing an exemplary multi-mode Zn(II)-selective emission-ratiometic probe in accordance with various embodiments of the present disclosure.

FIG. 5A illustrates spectral changes of probe (10 μM) in analytical grade methanol upon titration with Zn(II). (A) Absorption (UV-Vis) spectral changes upon saturation with Zn(II). (B) Fluorescence emission response upon saturation with Zn(II) (excitation at 395 nm). Blue traces: free probe in methanol. Red traces: probe Zn(II) complex. Inset: fluorescence intensity at 520 nm as a function of molar equivalents of Zn(II).

FIG. 5B illustrates spectral characteristics of probe (10 μM) in response to divalent metal cations in aqueous buffer (pH 7.0, 10 mM PIPES, 0.1 M KCl, 25° C.) containing 100 μM 4:1 DMPC:DMPG liposomes. (A) Fluorescence emission response upon saturation with Zn(II) (excitation at 405 nm). Inset: Fluorescence intensity at 520 nm as a function of molar equivalents of Zn(II). (B) Emission-ratiometric response towards selected divalent metal cations at 80% fractional saturation in the absence (black bars) and presence (white bars) of Zn(II). The emission ratio was calculated based on the integrated fluorescence intensity between 600 nm-520 nm and 440 nm-495 nm (excitation at 405 nm).

FIG. 5C illustrates photophysical properties of probe and its Zn(II)-complex in PIPES buffer containing 100 μM of 4:1 DMPC:DMPG liposomes (pH 7.0, 10 mM PIPES, 0.1 M KCl, 25° C.).

FIG. 6A illustrates exemplary fluorometric determination the Zn(II) complex stability constant of probe via competition titration with EGTA. (A) Probe (10 μM) was equilibrated with molar equivalent of Zn(OTf)₂ in chelexed PIPES buffer (pH 7.0, 10 mM PIPES, 0.1 M KCl, 25° C.) containing 100 μM 4:1 DMPC:DMPG liposomes and titrated with EGTA to a final concentration of 50 μM. The fluorescence spectra (excitation: 405 nm) were analyzed by non-linear least squares fitting. (B) Change in fluorescence response at 515 nm with the corresponding fit using the equilibrium system model shown in the table below.

FIG. 6B illustrates definition of equilibrium system employed for the determination of Zn(II)-binding affinity of probe in accordance with various embodiments of the present disclosure.

FIG. 7A illustrates the ratiometric imaging of labile Zn(II) in live mouse fibroblast 3T3 cells with probe by TPEM (excitation at 750 nm). (A) Left: Integrated emission intensities collected with 520 nm-600 nm (BP2) and 440 nm-495 nm (BP1) bandpass filters, respectively. Right: Intensity ratio images with R=BP2/BP1. (B) Left: Ratio images (BP2/BP1) before (prior to t=5 min) and after the addition of 50 μM ZnSO₄ and 5.0 μM pyrithione. Addition of TPEN at t=15 min resulted in reversal of the fluorescence intensity ratio. (C) Mean emission ratio of the cytoplasmic region averaged over 10 cells (P values calculated for n=10 using a two-tailed test). (D) Time course of the average ratio change of the mean emission ratio in the cytosol averaged over 10 cells.

FIG. 7B illustrates the ratiometric imaging of labile Zn(II) pools in live mouse fibroblast 3T3 cells with probe by laser confocal fluorescence microscopy (excitation at 405 nm). (A) Left: Integrated emission intensities collected with 520 nm-600 nm (BP2) and 440 nm-495 nm (BP1) bandpass filters, respectively. Right: Intensity ratio images with R=BP2/BP1. (B) Left: Ratio images (BP2/BP1) before (prior to t=5 mins) and after addition of 50 μM ZnSO₄ and 5.0 μM pyrithione. Addition of TPEN at t=15 mins resulted in the reversal of the fluorescence ratio. (C) Mean fluorescence ratio of the cytoplasmic region averaged over 10 cells (P values calculated for n=10 using a two-tailed test). (D) Time course of the average ratio change for mean emission ratio in the cytosol averaged over 10 cells.

### DETAILED DESCRIPTION OF THE INVENTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents that operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

As used herein, “compound A,” “probe,” “Zn(II) probe,” and “probe 8” are interchangeable. As used herein, “probe” refers to the inventive compounds described herein, and unless otherwise specified, “probe” may describe the compounds in the free state, bound/complexed to zinc or any other metal, and/or further linked or conjugated to any other compound, linker, ligand, and the like. For example, and not limited, “Zn probe” can refer to, e.g., compounds included in general Formula I and/or Formula II. Additionally, by way of example only “Zn-probe complex” can include Zn(II)-Formula I complex, Zn(II)-Formula II complex, Zn(II)-selective emission ratiometric probe, and/or Zn(II)-Formula 2 complex.

The term “optionally substituted” or “substituted” means that the referenced group may be substituted with one or more additional group(s) individually, including various known protecting groups. The protecting groups that may form the protected derivatives of the above substituents are known to those of skill in the art and may be found in references such as Greene and Wuts. Unless otherwise specified, any of the substituents described herein can be substituted or unsubstituted. For example, “alkyl” can include, e.g., propyl or substituted propyl (e.g., propyl bromide).

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

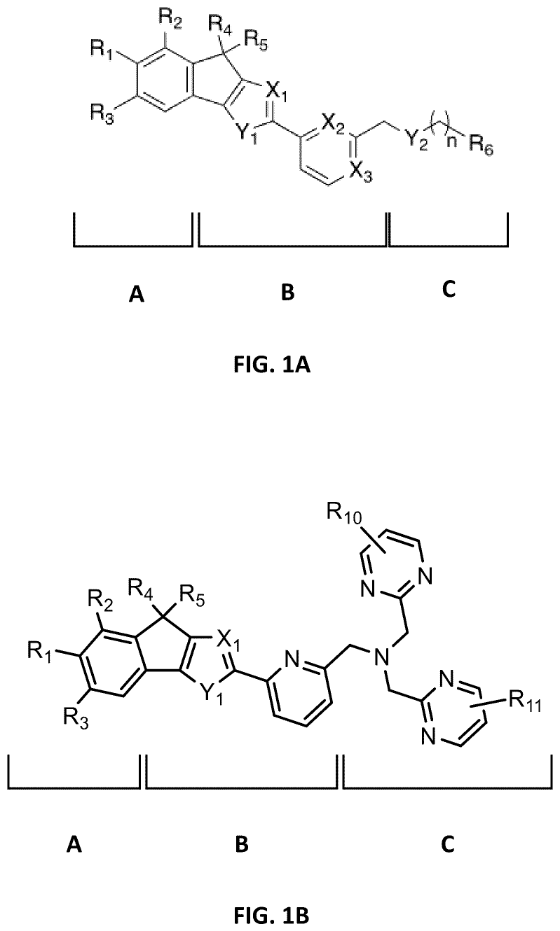

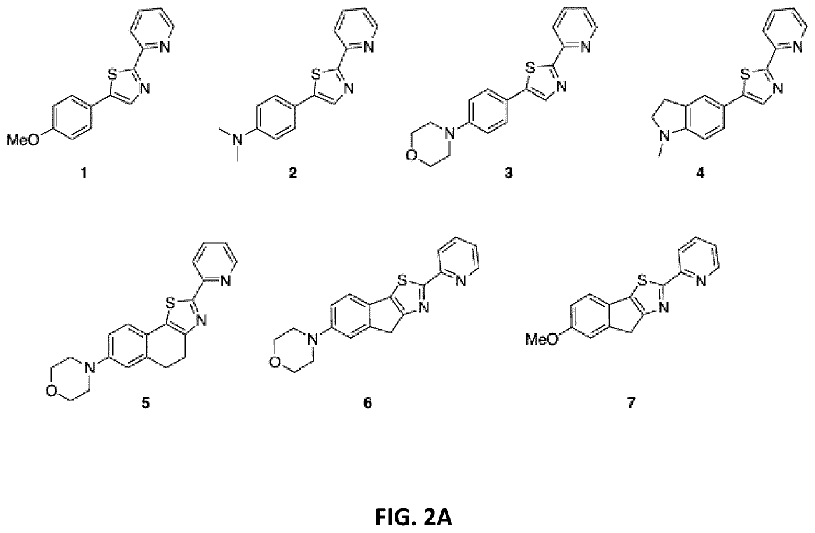

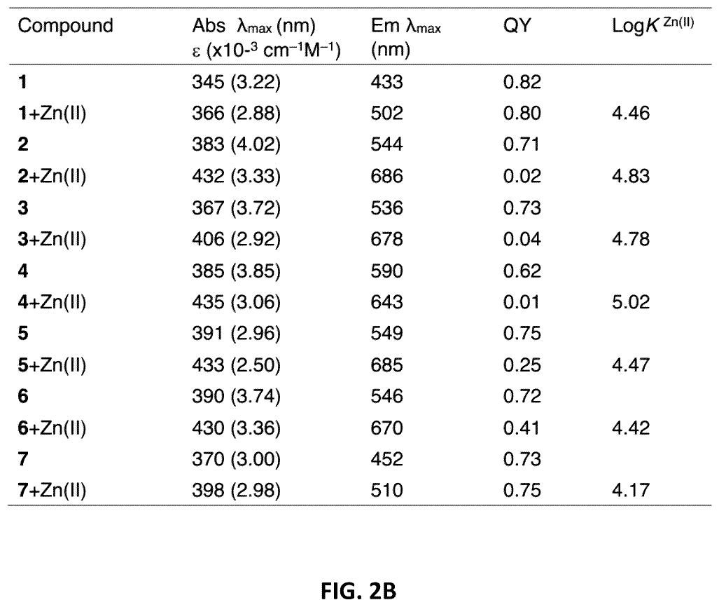

FIGS. 1A-1B illustrate schematics of exemplary compounds configured for visible-light excitation in accordance with various embodiments of the present disclosure. Component A can generally comprise any aryl moiety that imparts electron-donating properties and allows for further functionalization (e.g., to enhance water-solubility or to attach a linker for conjugation to a biomolecule). In various embodiments, Component A can be selected from a substituted or unsubstituted aryl and a substituted or unsubstituted heteroaryl. In some embodiments, Component A may be a pyridine ring, such that Component A may be a six-membered ring with five carbon atoms and one nitrogen atom. The nitrogen atom in the pyridine ring contributes to its aromaticity and also imparts different chemical properties compared to a simple aryl ring. In various embodiments, formula I and/or formula II may comprise a conformationally locked core configured to be substituted with a donor moiety, such that the substituted donor moiety is configured to fuse to a pyridine ring. The pyridine ring may be configured to act as an acceptor. The modification of formula I and/or formula II may be configured to tune the spectral characteristics, attach functional groups for bioconjugation with proteins, immobilization on solid support, tailor an affinity for a specific application, or a combination thereof. Formula I and/or Formula II may be configured to exhibit an absorption band around 405 nm for linear excitation with a laser or visible light and/or for non-linear two-photon excitation above 720 nm.

In some embodiments, R₁, R₂, and R₃ of Formula I and/or Formula II can be each independently selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl. Each R₁, R₂, and R₃ can be unsubstituted or substituted. Without wishing to be bound by theory, it is thought that R₁, which is in the para-position with respect to the pi-bridging component (Component B) can be critical for the photophysical properties of the compound. Accordingly, in some embodiments, R₁ can include an amine derivative to increase the electron-donating properties. Moreover, in some embodiments, R₁ can be selected from —OCH₃, —OCH₂COOR₉, —OCH₂CH₂COOR₉, —OCH₂CH₂CH₂SO₃R₉, —NH₂, —NHR₉, —N(R₉)₂. In various embodiments, R₉ from selected from hydrogen, —COR₈, —SO₂—R₈, substituted or unsubstituted alkyl, substituted or unsubstituted aryl, substituted or unsubstituted heteroalkyl, substituted or unsubstituted heteroalkenyl, substituted or unsubstituted heteroalkynyl, and substituted or unsubstituted heteroaryl. In various embodiments, —N(R₉)₂, may include amides and imides as donors, triazoles, alkyl substituents with active esters, sulfonamides, or vinyl sulfonamide and acrylamide for bioconjugation.

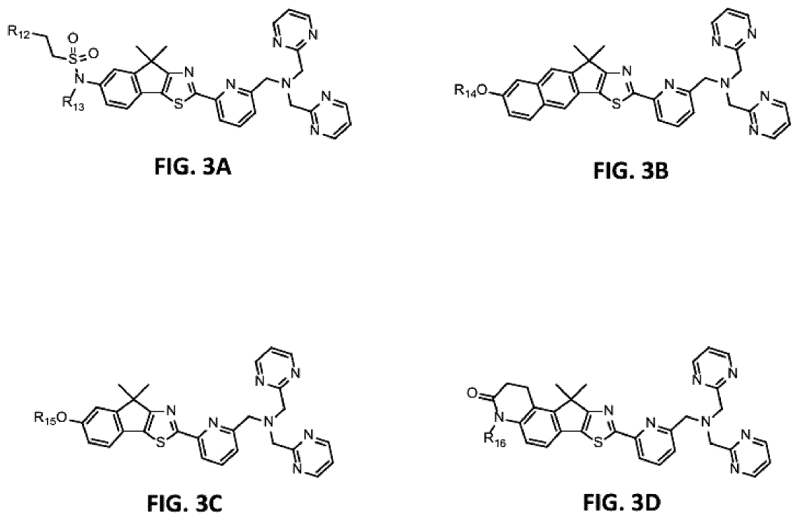

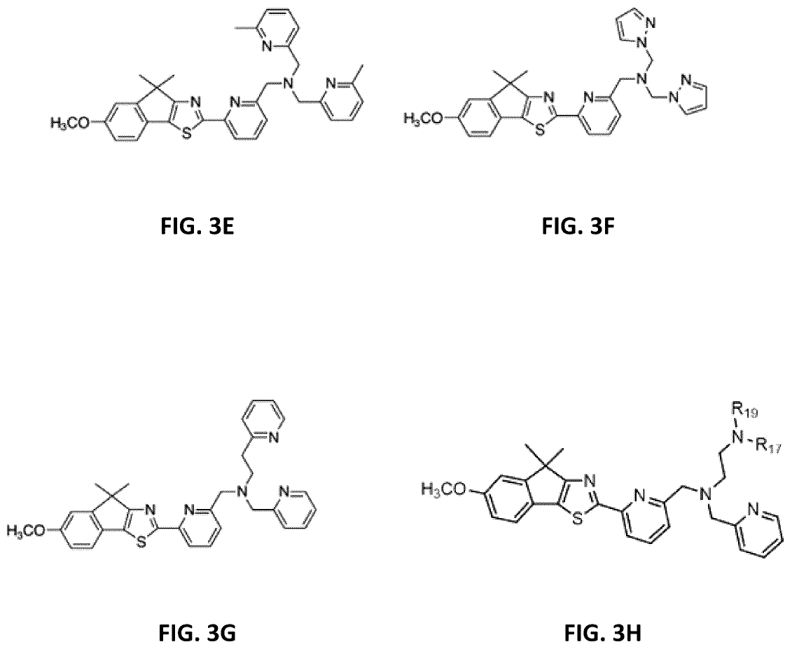

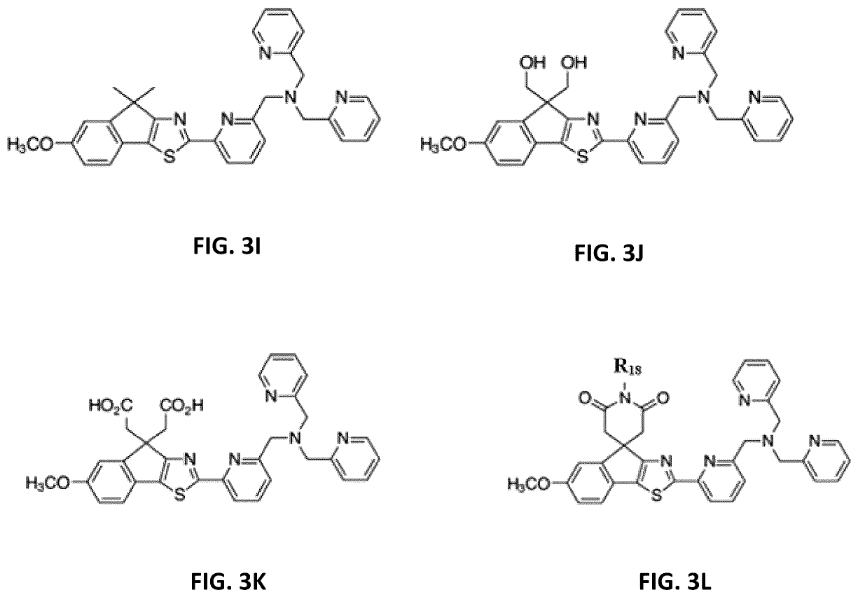

In various embodiments, R₁ may be selected from C₁₋₆-alkoxy, R₂ and R₃ are hydrogen. In other embodiments, R₁ and R₂ together form heterocycloalkyl, R₃ may be hydrogen. In an exemplary embodiment R₁ and R₃ together may form an aryl, and R₂ may be hydrogen. In one or more embodiments, R₁ and R₂ can join together to form a donor selected from substituted or unsubstituted heterocycloalkyl, substituted or unsubstituted heterocycloalkenyl, and substituted or unsubstituted heteroaryl. In other embodiments, R₁ and R₃ can join together to form a donor selected from substituted or unsubstituted heterocycloalkyl, substituted or unsubstituted heterocycloalkenyl, and substituted or unsubstituted heteroaryl. FIGS. 3A-3D illustrate exemplary donor variations of an exemplary multi-mode Zn(II)-selective emission-ratiometric probe in accordance with various embodiments of the present disclosure.

Component B can generally comprise any pi-system moiety that bridges Component A (aryl system) and Component C (Zn(II)-chelator moiety). In various embodiments, at least a portion of Component B (e.g., pyridine ring) may be a portion of the chelator and may be configured to serve as at least a portion of the electron-acceptor. Component B can be a conjugated system that can include delocalized pi electrons across all the adjacent aligned p-orbitals in the pi-bridging moiety. Such extension of the pi-conjugation length typically produces a bathochromic (i.e., red) shift in the absorption and emission spectrum. Accordingly, in some embodiments, Component B can be any aryl or heteroaryl moiety including, but not limited to, thiazole, oxazole, imidazole, thiophene, furan, or pyrrole. X₁, X₂, and X₃ are independently selected from —CH— and —N—. In some embodiments, Y₁ can be selected from —O—, —S—, —N(R₄)—, and —C(R₄)(R₅)—. In various embodiments, R₄ and R₅ are each independently selected from hydrogen, substituted or unsubstituted alkyl, alkenyl, alkynyl, cycloalkyl, cycloalkenyl, aryl, heteroalkyl, heteroalkenyl, heteroalkynyl, heterocycloalkyl, heterocycloalkenyl, and heteroaryl. In some embodiments, R₄ and R₅ join together to form a moiety selected from cycloalkyl, cycloalkenyl, aryl, heterocycloalkyl, heterocycloalkenyl, and heteroaryl. FIGS. 3J-3L illustrate exemplary affinity tunings of an exemplary multi-mode Zn(II)-selective emission-ratiometric probe in accordance with various embodiments of the present disclosure.

With reference to FIG. 3L, in various embodiments, R₁₈ may be selected from a group consisting of hydrogen, substituted or unsubstituted heterocycloalkyl, substituted or unsubstituted heteroalkyl, and substituted or unsubstituted heteroaryl.

Component C can generally comprise any moiety that will selectively bind to Zn(II) to form a fluorescent Zn(II)-probe complex with a red-shifted emission maximum relative to the unbound probe. In other words, as defined herein, “selectively” means that the resulting bound complex exhibits an emission maximum that is red-shifted by at least 10 nm relative to the free probe whereas other metal ions either do not elicit a shift or quench the fluorescence emission. In various embodiments, n may be 1, 2, or 3. In various embodiments, R₆ may be selected from C₁₋₃-alkyl optionally substituted with —OR₈, —COOH, —COOR₈, —NHCOR₈, —NH₂, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl. As depicted in FIG. 1A, Y₂ may be selected from —NR₇, —S—, and —O—. In various embodiments, R₇ is selected from C₁₋₃-alkyl optionally substituted with —OR₈, —COOH, —COOR₈, —NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl. As depicted in FIG. 1A, with reference to formula I, R₈ is selected from hydrogen, substituted or unsubstituted alkyl, substituted or unsubstituted aryl, substituted or unsubstituted heterocycloalkyl, substituted or unsubstituted heteroalkyl, and substituted or unsubstituted heteroaryl. In various embodiments, R₉ may be independently selected from hydrogen, —COR₈, —SO₂—R₈, substituted or unsubstituted alkyl, substituted or unsubstituted aryl, substituted or unsubstituted heteroalkyl, substituted or unsubstituted heteroalkenyl, substituted or unsubstituted heteroalkynyl, and substituted or unsubstituted heteroaryl.

As depicted in FIG. 1B, with reference to formula II, in various embodiments, R₁₀ and R₁₁ can be each independently selected from —H, —CH₃, —OR₈, —CF₃, —F, —Cl, —CN, —COOR₈, and —SO₃R₈. In various embodiments, R₈ may be selected from hydrogen, substituted or unsubstituted alkyl, substituted or unsubstituted aryl, substituted or unsubstituted heterocycloalkyl, substituted or unsubstituted heteroalkyl, and substituted or unsubstituted heteroaryl. FIGS. 3I-3L illustrate exemplary bridge modifications of an exemplary multi-mode Zn(II)-selective emission ratiometric probe in accordance with various embodiments of the present disclosure.

With reference to FIGS. 3E-3I, in various embodiments, R₁₇ and R₁₉ may be each independently selected from C₁₋₃-alkyl optionally substituted with —OR₈, —COOH, —COOR₈, —NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl.

Accordingly, in some embodiments, the disclosed probes can be configured to bind to zinc(II) (Zn(II)) to form a fluorescent Formula I-Zn(II) complex. In some embodiments, the maximum absorption wavelength of the Formula I-Zn(II) complex can be from about 380 nm to about 550 nm, and about 10 nm to about 60 nm greater than the maximum absorption wavelength of the free compound of Formula I (i.e., the unbound compound). In some embodiments, the maximum emission wavelength of the Formula I-Zn(II) complex is from about 410 nm to about 700 nm, and about 10 nm to about 60 nm greater than the maximum emission wavelength of the free compound of Formula I (i.e., the unbound compound).

### Exemplary Methods

The probe compounds described herein can be used in a wide variety of metal detecting methods, including, but not limited to UV-vis absorption spectroscopy, steady state fluorescence spectroscopy, time-resolved fluorescence spectroscopy, fluorescence microscopy, laser confocal fluorescence microscopy, two-photon excitation microscopy, light-sheet microscopy, or flow-cytometry. An embodiment of the present disclosure can be a method of detecting zinc in a sample comprising the steps of: (1) treating the sample with the Zn probe, for example, the Zn probe can comprise or consist of a compound of Formula I, Formula II, and/or a derivative thereof:

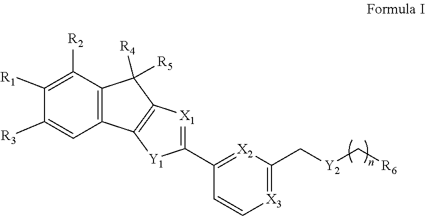

and/or a compound of Formula II

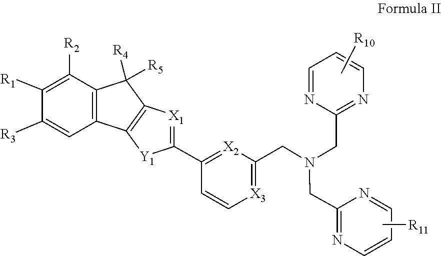

(2) detecting a light emission from the Zn probe upon excitation at a suitable wavelength, the light emission comprising a first wavelength emission range and a second wavelength emission range, wherein the first wavelength emission range intensity may be dominated by the fluorescence of the with an unbound Zn probe and the second wavelength emission range intensity may be dominated by the fluorescence of the Z(II)-bound Zn probe complex. (3) comparing the integrated intensity of the first wavelength emission range to the integrated intensity of the second wavelength emission range to determine the fractional saturation of the compound with Zn(II), from which the concentration or activity of zinc in the sample can be determined after appropriate calibration.

In some embodiments, the method can further comprise excitation of the Zn probe at a wavelength range associated with the absorption of the of the compound of Formula I, Formula II, Zn probe, the Zn(II)-probe complex, Zn(II)-Formula I complex, Zn(II)-Formula II complex, or a combination thereof. In some embodiments where the excitation wavelength range is only associated with the Zn(II)-probe complex, the detected light emission can be associated only with the Zn(II)-probe complex. In one or more embodiments, the linear excitation wavelength range can be above 380 nm. In some embodiments, the linear excitation wavelength range can be from about 350 nm to about 420 nm, from about 370 nm to about 440 nm, from about 390 nm to 460 nm, from about 410 nm to about 510 nm, from about 430 nm to about 530 nm, and from about 430 nm to about 550 nm. In various embodiments, a compound of Formula I, Formula II, and/or a derivative thereof (e.g., probe 8, Zn(II) complex) may be configured to exhibit a fluorescence emission maximum between about 420 to 650 nm, such that the emission maximum of the Zn(II) complex of the compound is from about 10 nm to about 60 nm greater than the emission maximum the compound alone As such, the two-photon excitation wavelength range can be from about 720 nm to about 950 nm. In one or more embodiment, the two-photon excitation wavelength range can start at 720 nm. In various embodiments, a compound of Formula I, Formula II, and/or a derivative thereof (e.g., probe 8) may be configured to shift the emission maximum by about 10 nm to about 60 nm upon formation of a complex with Zn(II). A person of ordinary skill in the art would know that various lasers can be employed as single wavelength excitation sources, and that the two-photon excitation wavelength of a femtosecond-pulsed Ti-Sapphire laser can be tuned to the optimal wavelength.

In some embodiments, the detection method can be two-photon excitation microscopy. In some embodiments, the detection method can be traditional linear excitation fluorescence microscopy. In some embodiments, the detection method can be confocal laser scanning microscopy. In one or more embodiments, the linear excitation wavelength range can be above 380 nm. In some embodiments, the linear excitation wavelength range can be from about 350 nm to about 420 nm, from about 370 nm to about 440 nm, from about 390 nm to 460 nm, from about 410 nm to about 510 nm, from about 430 nm to about 530 nm, and from about 430 nm to about 550 nm. As such, the two-photon excitation wavelength range can be from about 720 nm to about 950 nm. In one or more embodiment, the two-photon excitation wavelength range can start at 720 nm. A person of ordinary skill in the art would know that various lasers can be employed as single wavelength excitation sources, and that the two-photon excitation wavelength of a Ti-Sapphire laser can be tuned to the optimal wavelength.

In some embodiments, the light emission from the compound of Formula I, Formula II, and/or a derivative thereof can be from about 350 nm to about 400 nm, from about 370 nm to about 440 nm, from about 390 nm to 460 nm, from about 410 nm to about 510 nm, from about 430 nm to about 530 nm, from about 430 nm to about 550 nm, and about 720 nm to about 950 nm.

In various embodiments, the method may further comprise detecting Zn(II) by using ratiometric fluorescence analysis of the compound emission over two different wavelength ranges, by comparing the integrated emission intensity over the first wavelength to the integrated emission intensity over the second wavelength range. In one or more embodiments, the method may additionally comprise determining the concentration or activity of Zn(II) in the treated sample.

### Example 1. Synthesis of Compound 1

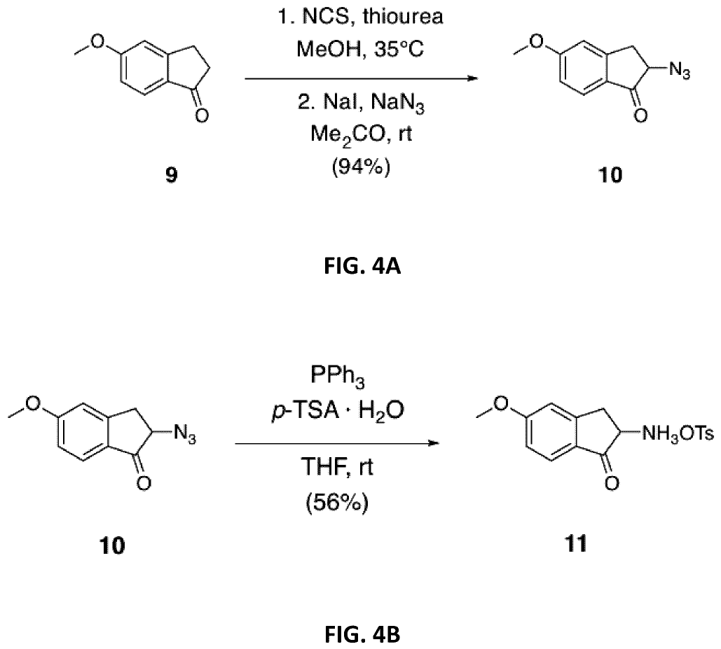

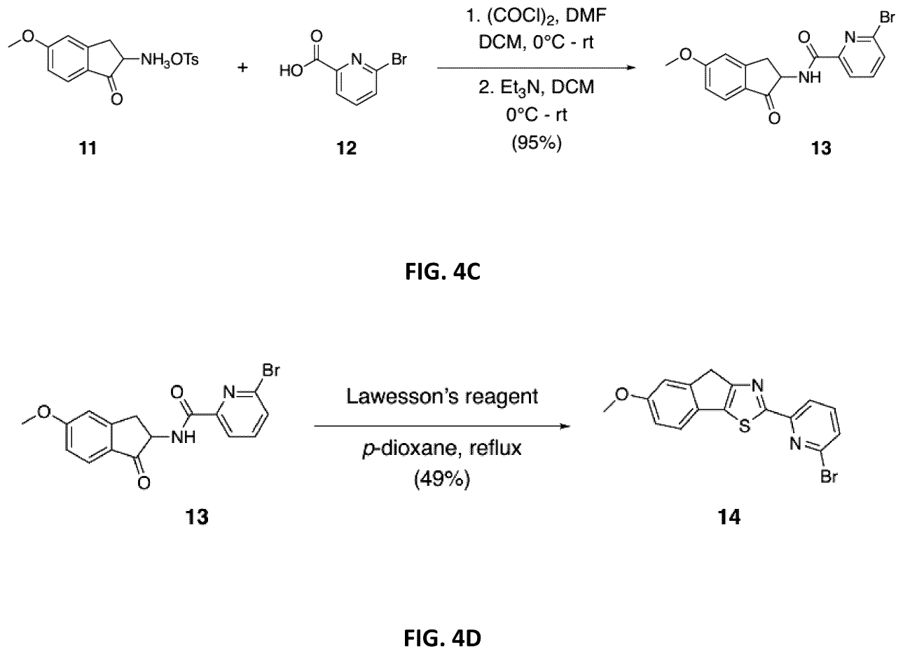

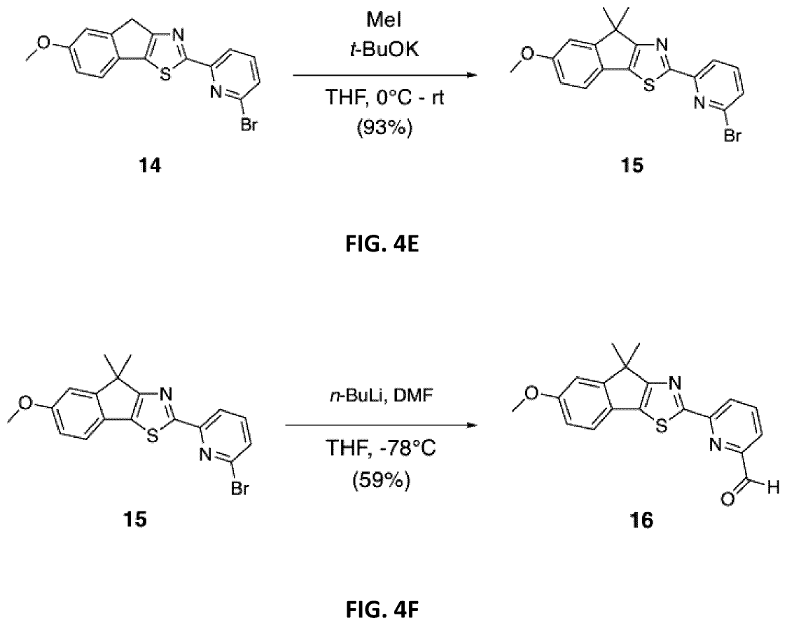

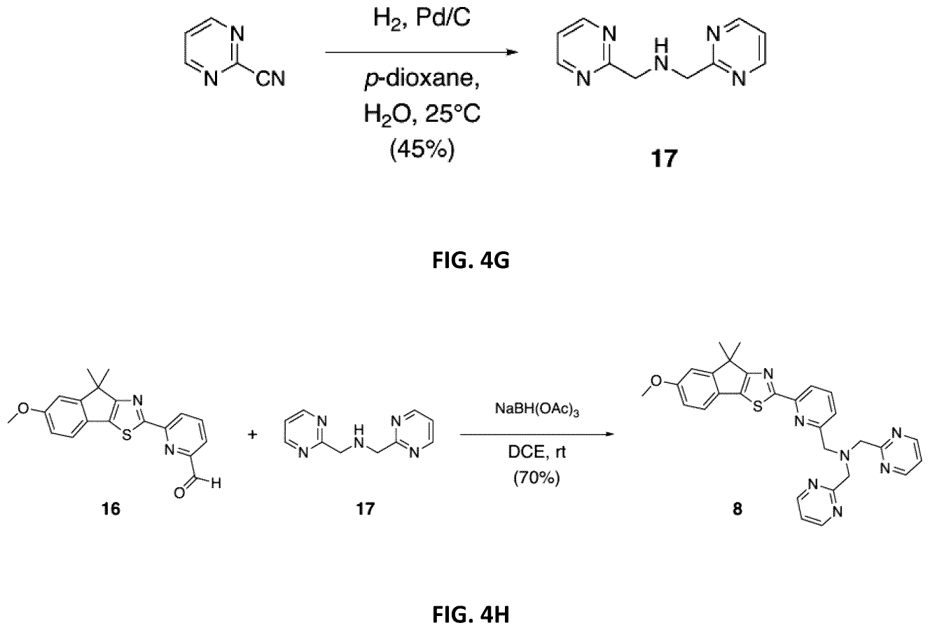

FIGS. 4A-4H illustrate portion of an exemplary synthetic scheme for accessing an exemplary multi-mode Zn(II)-selective emission ratiometric probe in accordance with various embodiments of the present disclosure. The Zn(II)-selective fluorescent probe 8 was synthesized from three building blocks: the interlocked 6-methoxy-4H-indeno[2,1-d]thiazole core, the 2-pyridinyl acceptor 12, and the bis(pyrimidin-2-ylmethyl)amine chelator moiety 17. Following a linear synthetic approach, commercially available 5-methoxy-1-indanone 9 was subjected to alpha-chlorination with a catalytic amount of thiourea. The resulting alpha-chloro ketone was substituted with azide employing modified Finkelstein reaction conditions, where an alpha-iodo group was generated in situ to lower the kinetic barrier. The alpha-amino ketone intermediate 11 was generated via Staudinger reaction in the presence of excess p-toluenesulfonic acid followed by amide coupling with 6-bromopicolinic acid 12. Tandem thionation-cyclization of intermediate 13 with Lawesson's reagent yielded the fused thiazole intermediate 14 with 49% yield. Alkylation of the methylene bridge with methyl iodide under mild conditions afforded intermediate 15, which was subjected to lithium-halogen exchange to generate in situ the reactive 2-pyridinyl lithium species followed by reaction with anhydrous N,N-dimethylformamide to yield the corresponding picolinaldehyde derivative 16 in 59% yield. Lastly, the chelator moiety was introduced via reductive amination of intermediate 16 with bis(pyrimidin-2-ylmethyl)amine 17 to yield the Zn(II)-selective fluorescent probe 8 in 70% yield.

With reference to FIG. 4A, to an oven-dried round bottom flask (250 mL) is added 5-methoxy-1-indanone (1.0 eq., 36.4 mmol, 5.90 g), N-chlorosuccinimide (1.2 eq., 43.7 mmol, 5.84 g), and thiourea (2 mol %, 0.728 mmol, 55 mg). Distilled MeOH (52 mL) is poured through a glass funnel. The reaction flask is capped with a rubber septum and purged with a steady stream of argon. The starting materials slowly dissolve at 35° C. under vigorous stirring, ensued by formation of colorless precipitation. The reaction progress is monitored via ¹H NMR. After two hours, the reaction mixture is cooled to room temperature, and concentrated under reduced pressure to a viscous slush. The reaction flask is transferred into an ice-bath. DI H₂O (100 mL) is poured into the crude product mixture upon rapid stirring. After 10 mins, the resulting colorless solid is separated via vacuum filtration, washed with DI water, and dried under vacuum over 15 hours to furnish the analytically pure product as a fluffy colorless powder (6.93 g, 96% yield). The product is used directly for the next step without further purification. ¹H NMR (500 MHz, chloroform-d) δ 3.25 (dd, J=17.6, 3.8 Hz, 1H), 3.73 (dd, J=17.6, 7.4 Hz, 1H), 3.90 (s, 3H), 4.54 (dd, J=7.4, 3.8 Hz, 1H), 6.87 (dd, J=2.3, 1.1 Hz, 1H), 6.96 (dd, J=8.6, 2.3 Hz, 1H), 7.76 (d, J=8.6 Hz, 1H). ¹³C NMR (101 MHz, chloroform-d) δ 37.78, 55.98, 56.17, 109.68, 116.53, 126.99, 127.12, 154.01, 166.58, 197.50. HRMS (ESI) m/z calculated for C₁₀H₉ClO₂ ([M+H]⁺) 197.0364. found 197.0364.

In various embodiments, to an oven-dried round bottom flask (250 mL) containing 2-chloro-5-methoxy-2,3-dihydro-1H-inden-1-one (1.0 eq., 35.2 mmol, 6.93 g), NaI (2.0 eq., 70.5 mmol, 10.56 g), and NaN₃ (2.0 eq., 70.5 mmol, 4.58 g) is added acetone (100 mL) under argon atmosphere. The pale-yellow heterogenous solution mixture is stirred rapidly at room temperature. If the starting material contained water, it would turn the solution purple and affect the reaction yield severely. After 20 hours, the brown reaction mixture is filtered. The filtrate is concentrated under reduced pressure to a viscous consistency. Under vigorous stirring, DCM (200 mL) is added to the dark brown mixture to precipitate the inorganic salts. The organic layer is separated via vacuum filtration. The filtrate is collected and concentrated under reduced pressure. The crude product is purified via column chromatography (silica gel, 50% EtOAc in hexanes) to yield the product 10 as a colorless solid (6.98 g, 98% yield). ¹H NMR (400 MHz, chloroform-d) δ 2.88 (dd, J=17.2, 4.4 Hz, 1H), 3.44 (dd, J=17.2, 8.0 Hz, 1H), 3.90 (s, 3H), 4.29 (dd, J=8.0, 4.4 Hz, 1H), 6.86 (d, J=2.2 Hz, 1H), 6.94 (dd, J=8.5, 2.2 Hz, 1H), 7.73 (d, J=8.5 Hz, 1H). ¹³C NMR (126 MHz, chloroform-d) δ 33.09, 55.80, 62.05, 109.68, 116.26, 126.46, 127.30, 154.33, 166.35, 199.66. HRMS (ESI) m/z calculated for C₁₀H₉N₃O₂ ([M+H]⁺) 204.0767. found 204.0767.

With reference to FIG. 4B, to a stirring homogenous solution of 2-azido-5-methoxy-2,3-dihydro-1H-inden-1-one 10 (1.0 eq., 24.9 mmol, 5.06 g) in anhydrous THF (125 mL) is slowly added PPh₃ (1.0 eq., 24.9 mmol, 6.53 g) and p-TSA-H₂O (3.0 eq., 74.7 mmol, 14.2 g) as a solid mixture. The reaction flask is capped with a rubber septum and purged with a steady stream of argon for 15 mins (with a bubbler for ventilation). The solution mixture is stirred rapidly overnight at room temperature. After 24 hours, the crude product is separated via vacuum filtration. The resulting light brown solid is washed with cold THF (30 mL) and dried under reduced pressure overnight to afford the product as a colorless powder (6.97 g, 64% yield). ¹H NMR (500 MHz, methanol-d₄) δ 2.36 (br s, 4H), 3.04 (dd, J=16.9, 5.3 Hz, 1H), 3.64 (dd, J=17.0, 8.3 Hz, 1H), 3.92 (s, 3H), 4.20 (dd, J=8.3, 5.3 Hz, 1H), 7.05 (dd, J=8.6, 2.3 Hz, 1H), 7.12-7.09 (m, 1H), 7.25-7.20 (m, 3H), 7.72-7.66 (m, 3H), 7.72 (d, J=8.6 Hz, 1H). ¹³C NMR (126 MHz, methanol-d₄) δ 21.34, 32.42, 55.12, 56.59, 111.13, 117.95, 126.97, 127.18, 128.06, 129.84, 141.70, 143.55, 155.69, 168.48, 198.78. HRMS (ESI) m/z calculated for C₁₀H₁₁NO₂ ([M+H]⁺) 178.0863. found 178.0862.

With reference to FIG. 4C, to a solution of 6-bromopicolinic acid 12 (1.0 eq., 19.6 mmol, 3.96 g) in anhydrous DCM (196 mL) is slowly added oxalyl chloride (1.5 eq., 29.4 mmol, 2.5 mL) with rapid stirring at 0° C., followed by the addition of a drop of DMF. The solution mixture slowly turned homogenous, and gas started evolving. A bubbler is used for ventilation, and the reaction is allowed to stir and slowly warm up to room temperature until it ceased effervescing. After 2 hours, the volatiles are removed under reduced pressure. The yellow waxy solid is dried under high vac for an hour and the crude 6-bromopicolinoyl chloride is directly used for the next step without further purification.

To a 250 mL round bottom flask containing 6-bromopicolinoyl chloride (˜19.6 mmol) in 196 mL anhydrous DCM is added 11 (1.0 eq., 19.6 mmol, 8.53 g). The reaction is transferred to an ice-bath and stirred under vigorously. After 15 mins, triethylamine (3.5 eq., 68.5 mmol, 9.6 mL) is slowly added. The reaction is allowed to stir at 0° C. and slowly warm up to room temperature until completion. After 6 hours, the purple-colored reaction is washed with DI water (100 mL×2). The organic layer was separated, dried over anhydrous Na₂SO₄, and filtered. The volatiles are removed under reduced pressure. The resulting viscous purple oil is immediately subjected to flash chromatography (silica gel, 30%-50% EtOAc in hexanes). The resulting off-white product is recrystallized in cyclohexane to yield 13 as a colorless crystalline solid (6.72 g, 95% yield). ¹H NMR (500 MHz, chloroform-d) δ 3.12 (dd, J=16.8, 5.4 Hz, 1H), 3.75 (dd, J=16.8, 8.2 Hz, 1H), 3.91 (s, 3H), 4.74 (ddd, J=8.2, 7.0, 5.4 Hz, 1H), 6.91 (dd, J=2.2, 1.0 Hz, 1H), 6.96 (dd, J=8.5, 2.2 Hz, 1H), 7.63 (dd, J=7.8, 1.0 Hz, 1H), 7.74-7.70 (t, J=7.8, 1H), 7.77 (d, J=8.5 Hz, 1H), 8.15 (dd, J=7.8, 1.0 Hz, 1H), 8.34 (d, J=6.9 Hz, 1H). ¹³C NMR (126 MHz, chloroform-d) δ 34.49, 55.75, 55.93, 109.74, 115.97, 121.31, 126.16, 128.08, 131.01, 139.62, 140.66, 150.44, 154.31, 163.42, 166.04, 200.45. HRMS (ESI) m/z calculated for C₁₆H₁₄O₃N₂Br ([M+H]⁺) 361.0182. found 361.0178.

With reference to FIG. 4D, to an oven dried 50 mL round bottom flask containing 13 (1.0 eq., 7.72 mmol, 2.79 g) and Lawesson's reagent (0.6 eq., 4.64 mmol, 1.88 g) is added anhydrous p-dioxane (15.4 mL) under argon atmosphere. The reaction flask is fitted with a condenser and purged with a steady stream of argon. The solution mixture is heated to 101° C. under vigorous stirring for 40 mins. The reaction mixture is cooled to room temperature and basified with 5% NaOH (100 mL). The red organic layer is separated, and the yellow aqueous layer was extracted with EtOAc until no longer blue fluorescent on TLC. The organic solvent extracts are combined and dried over anhydrous Na₂SO₄. The red organic layer is concentrated under reduced pressure to a viscous red oily residue. The crude product is precipitated with cold MeOH. The liquid layer is separated, dried under reduced pressure, and washed with MeOH repeated until all the product precipitated as an orange solid. The orange precipitations are combined and then purified via column chromatography (silica gel, 10%-20% EtOAc in hexanes) to afford the product 14 as a bright yellow solid (1.36 g, 49% yield). ¹H NMR (500 MHz, chloroform-d) δ 3.88 (s, 3H), 3.88 (br s, 2H), 6.93 (dd, J=8.3, 2.4 Hz, 1H), 7.16-7.13 (m, 1H), 7.45 (dd, J=7.8, 0.9 Hz, 1H), 7.47 (d, J=8.3 Hz, 1H), 7.62 (t, J=7.8 Hz, 1H), 8.09 (dd, J=7.8, 0.9 Hz, 1H). ¹³C NMR (126 MHz, chloroform-d) δ 34.31, 55.60, 111.89, 112.91, 117.96, 121.37, 127.98, 129.50, 139.08, 139.21, 141.57, 146.55, 152.86, 158.81, 163.04, 167.05. HRMS (ESI) m/z calculated for C₁₆H₁₂ON₂BrS ([M+H]⁺) 358.9848. found 358.9837.

With reference to FIG. 4E, to a rapidly stirred solution of 414 (1.0 eq., 3.74 mmol, 1.342 g) in anhydrous THF (18.5 mL) is added t-BuOK (3.0 eq., 11.21 mmol, 1.26 g as a solution in anhydrous THF, 18.5 mL) at 0° C. under argon atmosphere. The reaction is stirred at 0° C. for 10 min, followed by the addition of Mel (3.0 eq., 11.21 mmol, 0.7 mL). Precipitation starts to form immediately. The reaction is stirred and slowly warmed up to room temperature overnight. After 20 hours, the camel-colored solution mixture is diluted with DI water and neutralized with iN HCl. The organics are extracted with DCM (2×50 mL). the organic layers are combined and dried over anhydrous Na₂SO₄. The volatiles are removed under reduced pressure. The resulting brown solid is flushed through a short silica plug with 10%-15% EtOAc in hexanes and then recrystallized in MeOH to afford the product as a bright yellow solid (1.35 g, 93% yield). ¹H NMR (500 MHz, chloroform-d) δ 1.55 (s, 6H), 3.88 (s, 3H), 6.85 (dd, J=8.3, 2.4 Hz, 1H), 6.99 (d, J=2.4 Hz, 1H), 7.41 (d, J=8.3 Hz, 1H), 7.42 (dd, J=7.8, 0.9 Hz, 1H), 7.60 (t, J=7.8 Hz, 1H), 8.13 (dd, J=7.8, 0.9 Hz, 1H). ¹³C NMR (126 MHz, chloroform-d) δ 24.95, 45.16, 55.61, 109.69, 112.06, 118.24, 121.44, 127.00, 127.80, 135.71, 138.92, 141.43, 153.09, 157.36, 159.15, 167.18, 172.64. HRMS (ESI) m/z calculated for C₁₈H₁₆ON₂BrS ([M+H]⁺) 387.0161. found 387.0154.

With reference to FIG. 4F, to an oven-dried round bottom flask is equipped with a magnetic stir bar was charged with 15 (1.0 eq., 1.07 mmol, 415 mg,) and vacuumed and backfilled with argon (three times). Anhydrous THF (5.4 mL) is added via a syringe under argon atmosphere. The solution is cooled to −78° C. in a dry ice/acetone bath, followed by the addition of n-BuLi (2.5 M in hexanes) with a gas tight syringe. The resulting brown solution mixture is stirred at −78° C. for 20 mins, and anhydrous DCM (0.33 mL) is added to the solution mixture. The reaction is allowed to stir vigorously and slowly warm up to room temperature overnight. After 18 hours, saturated NH₄Cl solution is added slowly to quench the reaction. The solution mixture is diluted with EtOAc and washed with DI water. The aqueous layer is separated and extracted with more EtOAc until no longer blue fluorescent. The organic layers are combined and dried over anhydrous Na₂SO₄. The volatiles are removed under reduced pressure. The crude product mixture is purified via flash column chromatography (10%-15% EtOAc in hexanes) to afford the product as an orange solid, which is recrystallized in MTBE to a mustard yellow powder (213 mg, 59% yield). ¹H NMR (500 MHz, chloroform-d) δ 1.57 (s, 6H), 3.88 (s, 3H), 6.87 (dd, J=8.3, 2.4 Hz, 1H), 7.00 (d, J=2.4 Hz, 1H), 7.44 (d, J=8.3 Hz, 1H), 7.97-7.89 (m, 2H), 8.41 (dd, J=7.3, 1.7 Hz, 1H), 10.12 (s, 1H). ¹³C NMR (126 MHz, chloroform-d) δ 24.96, 45.19, 55.61, 109.72, 112.11, 121.15, 121.46, 123.69, 126.97, 135.68, 137.79, 152.39, 152.79, 157.42, 159.20, 167.73, 172.66, 193.18. HRMS (ESI) m/z calculated for C₁₉H₁₆N₂O₂S ([M+H]⁺) 337.1005. found 337.0999.

With reference to FIG. 4G, palladium (10%) on activated carbon (50% wet by H₂O, 2 mol %, 2.43 g) is added to a 250 mL round bottom flask containing 2-cyanopyrimidine (1.0 eq., 57.1 mmol, 6.0 g) and 125 mL of the p-dioxane: DI H₂O mixture (4:1 by volume). The flask is equipped with a 3-way stopcock adapter connected to an H₂ burette, which is filled with hydrogen gas. The flask is evacuated until gas bubbles start to evolve from the solvent, followed by immediate backfilling of hydrogen gas (repeated three times). The heterogenous solution mixture is stirred vigorously at room temperature while hydrogen gas (2.0 eq., 114 mmol, 2.76 L) is slowly consumed overnight. After complete consumption of the theoretical amount of H₂ gas, the flask is carefully purged with a steady stream of argon for 10 mins, and the palladium catalyst is filtered through a bed of celite and washed with p-dioxane (50 mL). The filtrate is collected and dried under reduced pressure to an amber-colored viscous oil. The oil residue is purified via column chromatography (silica gel, 1:4 EtOH:DCM) to afford 17 as an amber-colored oil (2.58 g, 45% yield). The oil residue 17 is fully dissolved in a MeOH/EtOH mixture in the presence of 2.0 eq. oxalic acid. The oxalate salt is subsequently brought to boiling and recrystallized from the solvent mixture, filtered, and dried under reduced pressure. ¹H NMR (D₂O, 400 MHz) δ 3.98 (s, 4H), 7.72 (dd, J=5.1, 1.5 Hz, 2H), 7.90 (s, 2H), 8.54 (dd, J=5.1, 0.7 Hz, 2H). ¹³C NMR (D₂O, 100 MHz) δ 54.9, 123.2, 123.5, 148.4, 150.0, 160.5, 172.7. EI-MS m/z 201 (10%, [M]⁺), 122 (23%,), 108 (100%), 94 (98%). EI-HRMS m/z calculated for C₁₀H₁₁N₅ (M+) 201.1014. found 201.1004.

With reference to FIG. 4H, to an oven dried round bottom flask with a magnetic stir bar is added 16 (1.1 eq., 0.297 mmol, 100 mg) and 17 (1.0 eq., 0.270 mmol, 110 mg). The flask is covered with a rubber septum and stored in the freezer until 17 solidified, followed by the addition of NaBH(OAc)₃ (1.5 eq., 0.4459 mmol, 94.5 mg). The reaction flask is transferred into an ice-bath and purged with a steady stream of argon for 10 mins. Anhydrous DCE is quickly added via a syringe, and the reaction is lifted out of the ice-bath and stirred at room temperature overnight. After 22 hours, the reaction is quenched with 1.0 M sodium citrate (1.0 mL) and diluted with DCM. The aqueous layer is separated and extracted with more DCM until no longer blue fluorescent on TLC. The organic layers are combined and dried over Na₂SO₄. The volatiles are removed under reduced pressure. The crude product mixture is purified via column chromatography (50%-75% acetone in DCM to remove impurity, and then neat acetone to elute the product) to afford a yellow glassy solid (109 mg, 70% yield). ¹H NMR (500 MHz, chloroform-d) δ 1.55 (s, 6H), 3.87 (s, 3H), 4.25 (s, 2H), 4.36 (s, 4H), 6.84 (dd, J=8.3, 2.4 Hz, 1H), 6.98 (d, J=2.4 Hz, 1H), 7.16 (t, J=4.9 Hz, 2H), 7.38 (d, J=8.3 Hz, 1H), 7.64 (d, J=7.7 Hz, 1H), 7.70 (t, J=7.7 Hz, 1H), 7.99 (d, J=7.7 Hz, 1H), 8.72 (d, J=4.9 Hz, 4H). ¹³C NMR (126 MHz, chloroform-d) δ 24.95, 45.12, 55.59, 59.90, 60.40, 109.65, 111.85, 117.69, 119.13, 121.05, 123.44, 127.46, 134.43, 137.11, 151.09, 157.06, 157.26, 158.78, 159.31, 168.57, 170.07, 172.36. HRMS (ESI) m/z calculated for C₂₉H₂₇N₇OS ([M+H]⁺) 522.2070. found 522.2059.

### Example 2. Steady-State Absorption and Fluorescence Properties of Compound I

PIPES buffer was prepared from piperazine-1,4-bis(2-ethansulfonic acid) disodium salt (TCI America) and volumetric standard KOH (Fluka). Oxalyl chloride, n-butyllithium solution (2.5 M in hexanes), tetrahydrofuran (DriSolv anhydrous grade with HT stabilizer), and anhydrous dichloroethane were purchased from Alfa Aesar. 1,2-dimyristoyl-sn-glycero-3-phosphocholine (DMPC) was purchased from Avanti Polar Lipids. Lawesson's reagent, 1,2-dimyristoyl-sn-glycero-3-phospho-(1′-rac-glycerol) (DMPG), ethyleneglycol-bis(β-aminoethyl)-N,N,N′,N′-tetraacetic acid (EGTA) and potassium tert-butoxide were purchased from TCI America. 5-Methoxy-1-indanone and 6-bromopicolinic acid were purchased from Combi-Blocks and used without further purification. All other reagents and materials were from standard commercial sources. All commercial reagents and solvents were used as received. Mass spectra were acquired at the Georgia Tech Mass Spectrometry facility.

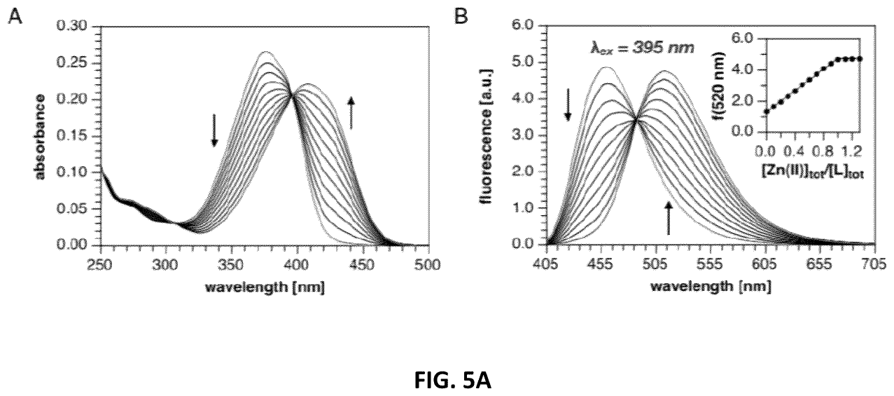

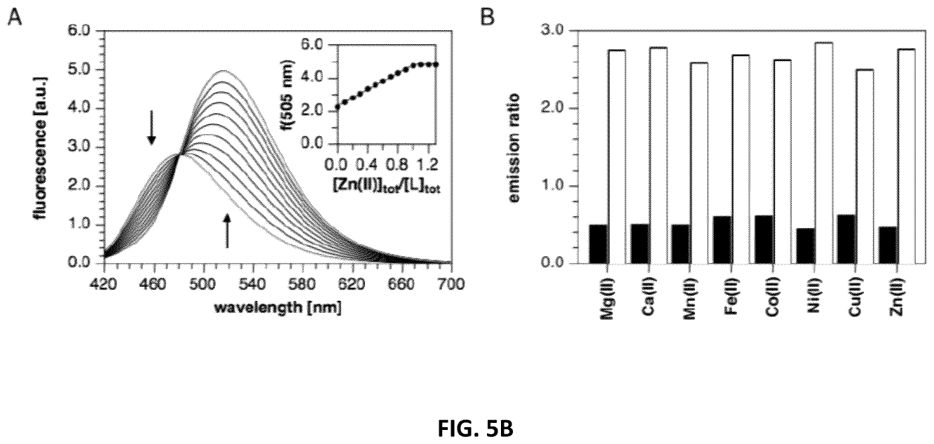

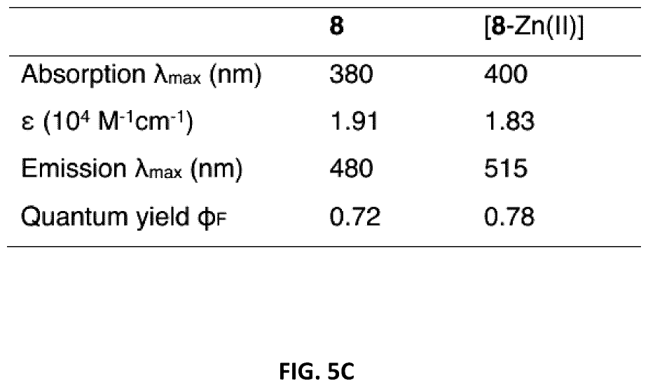

To prevent solution aggregation of the charge-neutral compounds exemplified by Formula I, Formula II, and/or a derivative thereof (e.g., for example probe 8) in PIPES buffer, the initial solution characterization was conducted in analytical grade MeOH. At micromolar concentrations, the coordination of Zn(II) resulted in a large bathochromic shift of both the absorption and emission spectrum, as evidenced by the molar ratio titration of probe 8 with Zn(OTf)₂ shown in FIGS. 5A-5C. Consistent with an increased ground- and excited state polarization upon Zn(II) complexation, the absorption maximum is red-shifted by 34 nm from 376 nm to 410 nm. Sharp isosbestic points at 306 nm and 395 nm, as well as saturation at an equimolar Zn(II) concentration are indicative of a well-defined solution equilibrium involving only the free probe 8 and a discrete Zn(II)-complex with 1:1 metal-ligand stoichiometry.

FIG. 5A illustrates exemplary spectral changes of Formula I probe, Formula II probe, and/or a derivative probe (e.g., for example probe 8) (10 μM) in analytical grade methanol upon titration with Zn(II). (A) of FIG. 5A, illustrates an exemplary absorption (UV-Vis) spectral changes upon saturation with Zn(II). (B) of FIG. 5A, illustrates an exemplary fluorescence emission response upon saturation with Zn(II) (excitation at 395 nm). Blue traces: free probe 8 in methanol. Red traces: probe 8 Zn(II) complex. Inset: fluorescence intensity at 520 nm as a function of molar equivalents of Zn(II).

Similarly, the fluorescence emission maximum of Formula I, Formula II, and/or a derivative thereof (e.g., for example probe 8) is red-shifted from 460 nm to 513 nm upon Zn(II)-binding. By exciting the probe at 395 nm, corresponding to the wavelength of the isosbestic point in the UV-vis spectrum, the observed emission intensities directly reflect the quantum yield ratio between the free probe 8 and the metal complex, thus revealing a balanced emission profile for TPEM imaging applications with an excitation wavelength around 750 nm. To provide a direct comparison with previous probes, probe 8 was also analyzed in aqueous buffer containing DMPC/DMPG liposomes that mimic the lipid bilayers present in cells.

FIG. 5B illustrates exemplary spectral characteristics of Formula I, Formula II, and/or a derivative thereof (e.g., for example probe 8) (10 μM) in response to divalent metal cations in aqueous buffer (pH 7.0, 10 mM PIPES, 0.1 M KCl, 25° C.) containing 100 μM 4:1 DMPC:DMPG liposomes. (A) of FIG. 5A illustrates an exemplary fluorescence emission response upon saturation with Zn(II) (excitation at 405 nm). Inset: Fluorescence intensity at 520 nm as a function of molar equivalents of Zn(II). (B) of FIG. 5A illustrates an exemplary emission-ratiometric response towards selected divalent metal cations at 80% fractional saturation in the absence (black bars) and presence (white bars) of Zn(II). The emission ratio was calculated based on the integrated fluorescence intensity between 600 nm-520 nm and 440 nm-495 nm (excitation at 405 nm).

To investigate the capability of Formula I, Formula II, and/or a derivative thereof (e.g., for example probe 8) as a dual-mode excitable Zn(II)-selective fluorescent indicator, fluorometric Zn(II)-titrations were performed with excitation at 405 nm. Comprehensive spectroscopic analysis of Formula I, Formula II, and/or a derivative thereof (e.g., for example probe 8) was performed in the presence of liposomes as a biological model-membranes composed of a 4:1 ratio of zwitterionic dimyristoyl phosphatidyl-glycerol (DMPC) and anionic dimyristoyl phosphatidylglycerol (DMPG). The probe was directly diluted from a DMSO stock solution into the pH 7.0 buffer containing 100 μM liposomes. Upon stepwise addition of Zn(II), the emission profile of probe 8 underwent a bathochromic shift from 480 nm to 515 nm. The emission traces revealed a sharp isoemissive point at 481, nm and saturation at an equimolar Zn(II) concentration confirmed a well-defined 1:1 Zn(II)-probe complex stoichiometry (FIG. 5B, (A)). Analogous to the emission profile of probe 8 in methanolic solution, the quantum yields of the free probe and its Zn(II)-probe complex are comparable in liposome solution as well, with 72% for the free probe and 78% for the Zn(II)-probe complex (e.g., Table 2, depicted in FIG. 5C). Thus, probe 8 and its Zn(II) complex exhibit a more balanced quantum yield ratio compared to other probes with 0.26 and 0.56 for the free and Zn(II)-bound form, respectively. The almost three-fold increase in quantum yield of the free probe 8 may be due to the absence of non-radiative deactivation pathways involving an excited state proton transfer (ESPT) to the para-substituted pyridine acceptor, further facilitated by the more basic isonicotinic acid groups compared to the pyrimidines of probe 8.

FIG. 5C illustrates an exemplary table providing exemplary photophysical properties of Formula I, Formula II, and/or a derivative thereof (e.g., for example probe 8) and its Zn(II)-probe complex in PIPES buffer containing 100 μM of 4:1 DMPC:DMPG liposomes (pH 7.0, 10 mM PIPES, 0.1 M KCl, 25° C.). The fluorescence response of the probe 8 (10 PM) was measured in the presence of 100 μM 4:1 DMPC:DMPG liposomes (pH 7.0, 10 mM PIPES, 0.1 M KCl, 25° C.) and an excess of selected biologically relevant divalent metal ions (FIG. 5A). To ensure that adventitious metal ions in the buffer do not interfere with the fluorescence spectrum of the metal-free probe 8, the PIPES buffer was treated with Chelex® resin. The ratiometric response of the probe 8 was evaluated in the presence of 0.8 molar equivalents of divalent transition metals, all of which resulted in quenching of the fluorescence. The remaining 20% of free probe remained unaffected by any of the quenching metal ions, and the saturation of the remaining 0.2 molar equivalents of the probe with Zn(II) produced a ratiometric response consistent with the emission response of the [(8)-Zn(II)] complex. Due to the lack of response towards Ca(II) and Mg(II) at equimolar concentrations, probe 8 was subjected to PIPES buffer containing 100 μM of 4:1 DMPC:DMPG liposomes (pH 7.0, 10 mM PIPES, 0.1 M KCl, 25° C.) in the presence of 2.0 mM of either metal ion. Neither Ca(II) nor Mg(II) resulted in any significant fluorescence change and the metal ions did not interfere with 1.0 molar-equivalents of Zn(II) relative to the probe, as manifested by a significant bathochromic shift similar to the one observed in above fluorescence molar ratio titration ((B) of FIG. 5B).

### Example 3. Zn(II)-Binding Affinity Measurements of Formula I

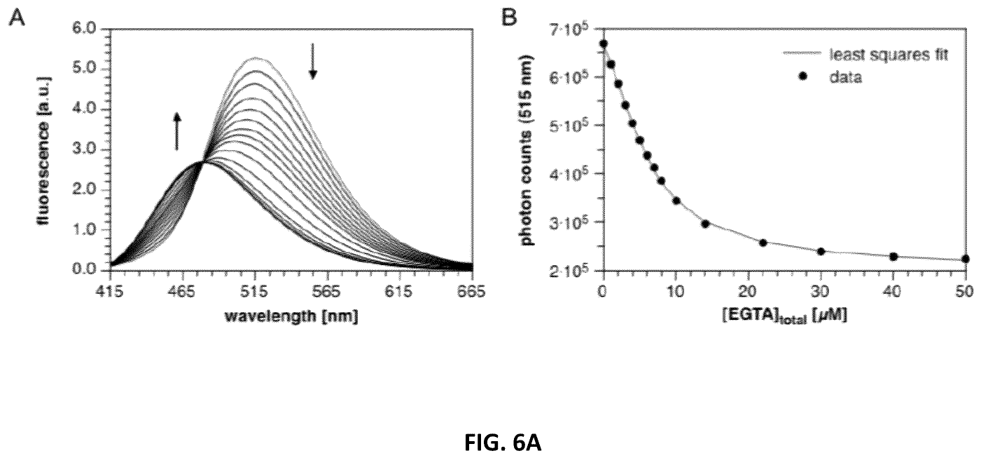

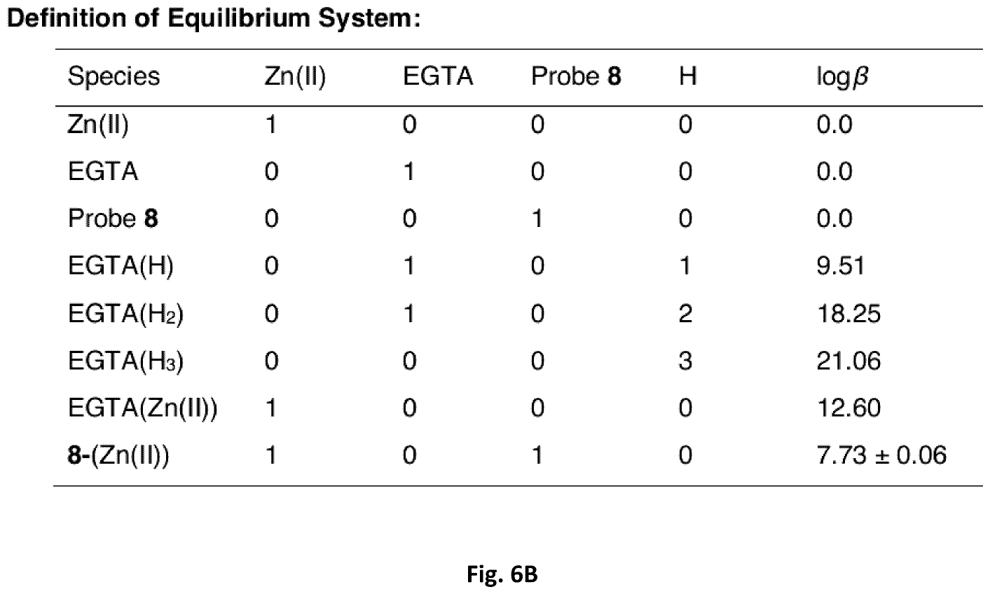

FIG. 6A illustrates the fluorometric determination of the Zn(II) complex stability constant of probe 8 via competition titration with EGTA. (A) of FIG. 6A illustrates an exemplary Formula I, Formula II, and/or a derivative thereof (e.g., for example probe 8) (10 μM) that was equilibrated with molar equivalent of Zn(OTf)₂ in chelexed PIPES buffer (pH 7.0, 10 mM PIPES, 0.1 M KCl, 25° C.) containing 100 μM 4:1 DMPC:DMPG liposomes and titrated with EGTA to a final concentration of 50 μM. The fluorescence spectra (excitation at 405 nm) were analyzed by non-linear least squares fitting. (B) FIG. 6A illustrates an exemplary change in fluorescence response at 515 nm with the corresponding fit using the equilibrium system model as defined in the table of FIG. 6B.

The binding affinity of Formula I, Formula II, and/or a derivative thereof (e.g., for example probe 8) was analyzed in PIPES buffer containing 100 μM of 4:1 DMPC:DMPG liposomes (pH 7.0, 10 mM PIPES, 0.1 M KCl, 25° C.). To accurately determine the Zn(II) affinity of probe 8, fluorometric titrations were performed with EGTA as a competing ligand with matching affinity (K_{d}=4.6 nM at pH 7.0, 25° C.). Non-linear least squares fitting of the fluorometric titration data provided an apparent stability constant of 7.73±0.06, corresponding to an apparent dissociation constant K_{d}=18.5 nM at pH 7.0 and an ionic background of 0.1 M KCl at 25° C. The binding affinity is over 400-fold weaker than chromis-1 acid, and approximately 10-fold lower than chromis-1 ester. The discrepancy in binding affinities is likely due to a combination of two factors. First, the two methyl groups attached to the rigidified fluorophore bridge might be responsible for some buildup of steric strain upon Zn(II) coordination, and second, the bis(pyrimidin-2-ylmethyl)amine was employed for its low pKₐ to avoid ESPT-induced fluorescence quenching. However, the reduced proton basicity goes hand-in-hand with a decreased Lewis basicity and thus weaker Zn(II) complexation.

### Example 4. Competition Titration and Zn(II) Stability Constant

A solution of Formula I, Formula II, and/or a derivative thereof (e.g., for example probe 8) was diluted from a 3.0 mM DMSO stock solution to a final concentration of 10 μM in Chelex®-treated PIPES buffer (10 mM, 0.1 M KCl, pH 7.0) containing 10 μM of Zn(OTf)₂ and 100 μM of liposomes (4:1 mixture of DMPC and DMPG). The resulting solution (3.0 mL total volume) was equilibrated at 25° C. under gentle stirring for 10 mins. After the addition of each aliquot of EGTA, the solution was allowed to equilibrate for 1 minute, and a fluorescence spectrum (excitation at 405 nm, spectral window 415-665 nm) was acquired. The titration was conducted in triplicate, and the data were analyzed by nonlinear least-squares fitting over the entire spectral range using the SPECFIT software package.

### Example 5. Quantum Yield Measurement

The quantum yields of the free Formula I, Formula II, and/or a derivative thereof (e.g., for example probe 8) was determined using quinine sulfate as a reference (D_{f}=0.546 in 1.0 N H₂SO₄). The quantum yield was derived from a 5-point measurement in Chelex®-treated PIPES buffer containing 100 μM of 4:1 DMPC:DMPG liposomes (pH 7.0, 10 mM PIPES, 0.1 M KCl, 25° C.). For the free compound 1 (e.g., for example probe 8 in FIG. 4H), the solutions were supplemented with 20 μM of EDTA (from a 3.0 mM stock solution in DI H₂O). The quantum yield of the probe 8-Zn(II) complex was independently determined under the same conditions in the presence of 1.1 molar equivalent of Zn(OTf)₂. The quantum yield of the Zn(II) complex was confirmed with fluorescence quantum yield ratio. The fluorescence quantum yields were calculated as an average of two independent measurements.

### Example 6. Molar Ratio Titration of Formula I, Formula II, and/or a Derivative

A solution of Formula I, Formula II, and/or a derivative thereof (e.g., for example probe 8) (10 μM) in analytical grade MeOH prepared in a quartz cuvette with a 1-cm pathlength was titrated with Zn(OTf)₂ (from a 0.3 mM aqueous stock solution) in 1.0 μM (0.1 molar equiv.) increments. After each aliquot of Zn(II), the solution was equilibrated for 1 minute by magnetic stirring, and an absorbance (spectral window: 250-500 nm) and fluorescence spectrum (excitation at 395 nm, spectral window 405-705 nm) were acquired. In another embodiment, a solution of Formula I, Formula II, and/or a derivative thereof (e.g., for example probe 8 in FIG. 4H) (10 PM) in Chelex®-PIPES buffer (10 mM, 0.1 M KCl, pH 7.0, 25° C.) containing 100 μM of liposomes (4:1 mixture of DMPC and DMPG) prepared in a quartz cuvette with a 1-cm pathlength was titrated with Zn(OTf)₂ (from a 0.3 mM aqueous stock solution) in 1.0 μM (0.1 molar equiv.) increments. After each aliquot of Zn(II), the solution was equilibrated for 1 minute by magnetic stirring, and a fluorescence spectrum (excitation: 405 nm, spectral window 420-700 nm) was acquired.

### Example 7. Metal-Ion Selectivity Measurements of Formula I, Formula II, and/or a Derivative

A solution of Formula I, Formula II, and/or a derivative thereof (e.g., for example probe 8) (10 μM) in Chelex®-PIPES buffer (10 mM, 0.1 M KCl, pH 7.0, 25° C.) containing 100 μM of liposomes (4:1 mixture of DMPC and DMPG) prepared in a quartz cuvette with a 1-cm pathlength. A fluorescence spectrum was acquired over the spectral window of 415-700 nm with excitation at 405 nm. Under magnetic stirring, the solution was supplemented with each divalent metal cation (8.0 μM, which corresponds to 80% fractional saturation of the probe, or 2.0 mM Ca(II) and Mg(II) ions), and a fluorescence spectrum was immediately acquired. The solution was supplemented with Zn(OTf)₂ (from a 0.3 mM stock in DI H₂O) to a final concentration of 2.0 μM. The fluorescence spectrum was acquired after 10 min equilibration to ensure complete formation of the probe 8 Zn(II)-complex. The emission ratio was calculated based on the integrated fluorescence intensity between 600 nm-520 nm (BP2) and 495 nm-440 nm (BP1). The metal cations were supplied as aqueous stock solutions of the following salts: MgCl₂, CaCl₂, Co(NO₃)₂, MnSO₄, FeSO₄, and CuSO₄. The FeSO₄ stock solution was prepared in 10 mM H₂SO₄ to avoid aerial oxidation.

### Example 8. Ratiometric Fluorescence Imaging with Formula I

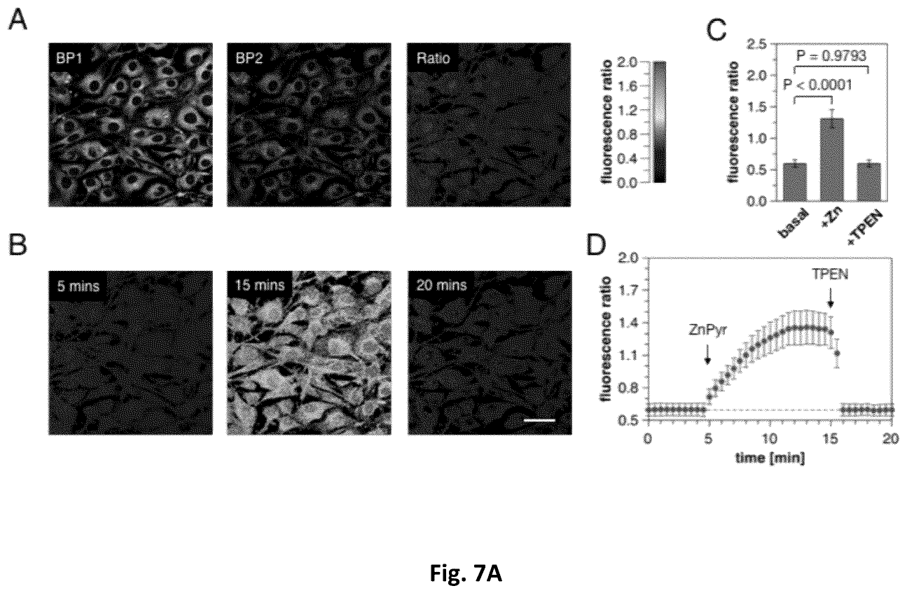

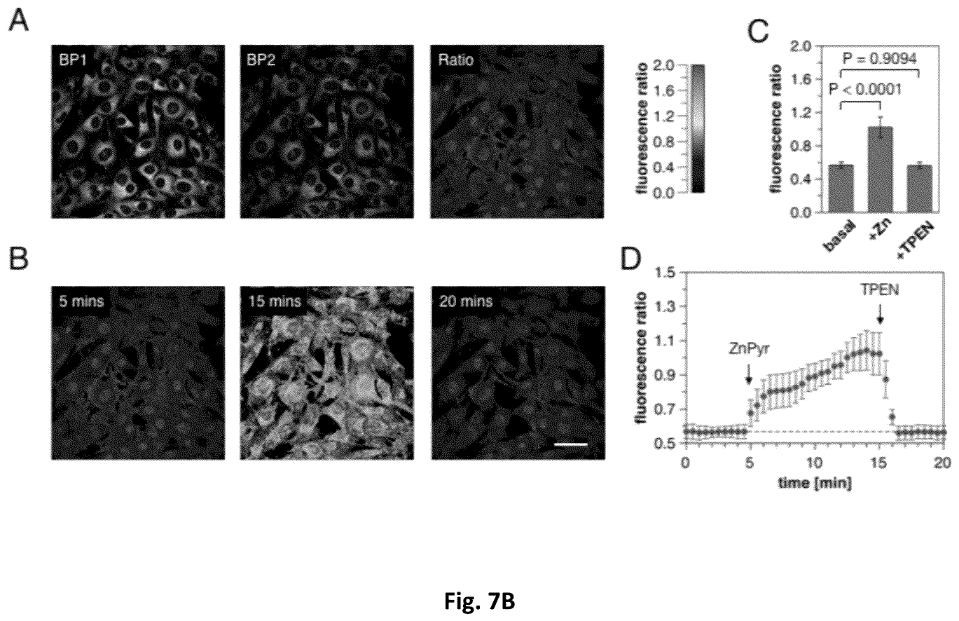

With reference to FIG. 7A, emission-ratiometric imaging of labile Zn(II) pools in live mouse fibroblast 3T3 cells with Formula I, Formula II, and/or a derivative thereof (e.g., for example probe 8) by TPEM (excitation at 750 nm). (A) of FIG. 7A Left: The emission was collected with 520 nm-600 nm (BP2) and 440 nm-495 nm (BP1) bandpass filters respectively. Right: Intensity ratio images with R=BP2/BP1. (B) of FIG. 7A Left: Ratio images (BP2/BP1) before (prior to t=5 min) and after the addition of 50 μM ZnSO₄ and 5.0 μM pyrithione. Addition of TPEN at t=15 min resulted the reversal of the initial of fluorescence ratio change. (C) of FIG. 7A Mean fluorescence ratio of the cytoplasmic region averaged over 10 cells (P values calculated for n=10 using a two-tailed test). (D) of FIG. 7A Time course of the average ratio change for the mean fluorescence ratio in the cytosol averaged over 10 cells.

To evaluate the Zn(II)-dependent ratiometric response of Formula I and/or Formula IA (e.g., for example probe 8) in live cells, imaging experiments were performed with live mouse fibroblasts (3T3) as model cell line. As indicated by the fluorescence micrographs (e.g., depicted in FIG. 7A), probe 8 was readily internalized by 3T3 cells as manifested by the bright fluorescence throughout the cytoplasm. The average fluorescence ratio of R=0.59±0.06 displayed a uniform fractional saturation throughout the cell population. Upon exposure of the cells to Zn(II) in combination with the membrane-permeable ionophore pyrithione, the fluorescence ratio rapidly increased more than 2-fold to yield an average ratio of R=1.35±0.16, indicating complexation of probe 8 with Zn(II) (e.g., depicted in (B) of FIG. 7A). Addition of the high-affinity membrane-permeable chelator TPEN reversed the fluorescence ratio back to the basal level yielding an average ratio of R=0.59±0.05

With reference to FIG. 7B, ratiometric imaging of labile Zn(II) pools in live mouse fibroblast 3T3 cells with Formula I and/or Formula IA (e.g., for example probe 8) by TPEM (excitation at 405 nm). (A) of FIG. 7B Left: Intensities acquired with 520 nm-600 nm (BP2) and 440 nm-495 nm (BP1) bandpass filters respectively. Right: Intensity ratio imagines with R=BP2/BP1. (B) of FIG. 7B Left: Ratio images (BP2/BP1) before (prior to t=5 min) and after the addition of 50 μM ZnSO₄ and 5.0 μM pyrithione. Addition of TPEN at t=15 min resulted the reversal of the initial fluorescence ratio change. (C) of FIG. 7B: Mean fluorescence ratio of the cytoplasmic region averaged over 10 cells (P values calculated for n=10 using a two-tailed test). (D) of FIG. 7B: Time course of the average ratio change for mean fluorescence ratio in the cytosol averaged over 10 cells.

To investigate whether Formula I, Formula II, and/or a derivative thereof (e.g., for example probe 8) may also be employed with one-photon excitation at 405 nm, the imagine experiment was repeated with a conventional confocal fluorescence microscope equipped with a 405 nm laser as the excitation source. Similar to the TPEM experiment, the 3T3 cells readily internalized probe 8 as indicated by the bright fluorescence throughout the cytoplasm. The averaged fluorescence ratio indicated a uniform fractional saturation with an average ratio of R=0.57±0.04. Upon exposure of the cells to Zn(II) and the membrane-permeable ionophore, the fluorescence ratio rapidly increased to yield an average ratio of R=1.04±0.12, which is lower than observed with two-photon excitation (FIG. 7B). The difference in fluorescence ratios is likely due to differences in the absorption cross sections for linear vs. non-linear excitation. In principle, the ratio response could be further tuned by adjusting the sensitivity of the bandpass detectors. Addition of the high-affinity membrane-permeable chelator TPEN reversed the fluorescence ratio back to the initial level under basal conditions yielding an average ratio of R=0.56±0.04. In summary, the emission-ratiometric imaging experiment demonstrated the capability of probe 8 for dual-mode excitation and the utility for both TPEM and laser confocal fluorescence microscopy to visualize labile Zn(II) fluxes in live cells.

In various embodiments, mouse fibroblast (3T3) cells were grown on poly-L-lysine treated glass bottom culture dishes (MatTek) to 90% confluency in DMEM growth media supplemented with 10% bovine calf serum, and 1% penicillin-streptomycin. Before imaging, the media was replaced with pre-warmed colorless serum-free DMEM (1% penicillin-streptomycin, and 1% sodium pyruvate) containing 1.0 μM of probe 8. The cells were incubated at 37° C. under an atmosphere of humidified air containing 5% CO₂ for 15 mins. The incubation solution was replaced with prewarmed colorless serum free DMEM. Images were acquired at 37° C., 8% humidity, and 5% CO₂ using a Zeiss LSM NLO 710 microscope equipped with a femtosecond pulsed Ti:sapphire laser and a blue 405 nm laser. Scanning fluorescence micrographs were acquired with excitation at 405 nm or 750 nm and the emission was simultaneously collected with bandpass ranges of 495-440 nm (BP1) and 600-520 nm (BP2). Image J was used to evaluate the change in fluorescence emission ratio over time as previously described. The emission ratio for each region of interest was averaged at each time point.

TPEN solid was fully dissolved in anhydrous DMSO to a concentration of 3.0 mM. The stock solution was further diluted to 1.0 mM in pre-warmed phenol-red-free DMEM media before each imaging experiments. The ZnSO₄ stock solution was prepared in DI H₂O and the pyrithione stock solution was prepared in anhydrous DMSO to a concentration of 3.0 mM. The stock solution of Zn(II)/pyrithione was prepared and diluted with phenol red free DMEM. To determine the limiting emission intensity ratios Rₘᵢₙ and Rₘₐₓ, mouse fibroblast cells were supplemented with Zn(II)-pyrithione (50 μM ZnSO₄ and 5.0 μM pyrithione), and then TPEN (100 μM).

The design of ratiometric fluorescence probes (e.g., Formula I, Formula II, and/or a derivative thereof, for example probe 8) for detecting trace metal ions in complex biological systems remains a significant challenge. Systematic optimization of our first-generation emission-ratiometric fluorescent probe chromis-1 yielded a versatile dual-mode probe for use in a wide range of fluorescence detection instrumentation. Confirmed by model studies, structural rigidification of the chromis-1 D-π-A core produced a large bathochromic shift in the absorption spectrum in both the free Formula I, Formula II, and/or a derivative thereof (e.g., for example probe 8) and the Zn(II)-bound complex without compromising the fluorescence emission ratio, quantum yields, and emission maxima.

In summary, the present description has successfully demonstrated an efficient synthesis of Formula I, Formula II, and/or a derivative thereof (e.g., for example probe 8) as a first implementation of the outlined design principle. Spectrophotometric analysis of Formula I, Formula II, and/or a derivative thereof (e.g., for example probe 8) revealed discrete 1:1 binding stoichiometry with Zn(II) in both methanolic solution and PIPES buffer containing 100 μM of 4:1 DMPC:DMPG liposomes (pH 7.0, 10 mM PIPES, 0.1 M KCl, 25° C.). The quantum yields of the free Formula I, Formula II, and/or a derivative thereof (e.g., for example probe 8) and Zn(II) complex are well matched in liposomes, as opposed to the chromis-1 ester. The modular molecular architecture of the probes provides opportunities for further tuning of the probe properties for specific applications beyond TPEM.

While certain embodiments of the disclosed technology have been described in connection with what is presently considered to be the most practical embodiments, it is to be understood that the disclosed technology is not to be limited to the disclosed embodiments, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain embodiments of the disclosed technology, including the best mode, and also to enable any person skilled in the art to practice certain embodiments of the disclosed technology, including making and using any devices or systems and performing any incorporated methods. The patentable scope of certain embodiments of the disclosed technology is defined in the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A compound according to Formula I: 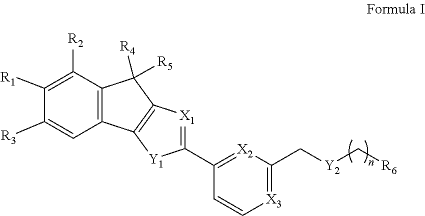
wherein:
n is 1, 2, or 3;
Y₁ is selected from —O—, —S—, —N(R₄)—, and —C(R₄)(R₅)—;
Y₂ is selected from —NR₇, —S—, and —O—; wherein R₇ is selected from C₁₋₃-alkyl optionally substituted with —OR₈, —COOH, —COOR₈, NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl;
X₁, X₂, and X₃ are independently selected from —CH— and —N—;
R₁ is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, substituted or unsubstituted heteroaryl, —OCH₃, —OCH₂COOR₉, —OCH₂CH₂COOR₉, —OCH₂CH₂CH₂SO₃R₉, —NHR₉, and —N(R₉)₂;
R₂ is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl; or in the alternative, R₁ and R₂ join together to form a donor selected from substituted or unsubstituted heterocycloalkyl, substituted or unsubstituted heterocycloalkenyl, and substituted or unsubstituted heteroaryl;
R₃ is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl; or in the alternative, R₁ and R₃ join together to form a donor selected from substituted or unsubstituted heterocycloalkyl, substituted or unsubstituted heterocycloalkenyl, and substituted or unsubstituted heteroaryl;
R₄ and R₅ are each independently selected from hydrogen, substituted or unsubstituted alkyl, alkenyl, alkynyl, cycloalkyl, cycloalkenyl, aryl, heteroalkyl, heteroalkenyl, heteroalkynyl, heterocycloalkyl, heterocycloalkenyl, and heteroaryl, R₄ and R₅ join together to form a moiety selected from cycloalkyl, cycloalkenyl, aryl, heterocycloalkyl, heterocycloalkenyl, and heteroaryl;
R₆ is selected from C₁₋₃-alkyl optionally substituted with —OR₈, —COOH, —COOR₈, —NHCOR₈, —NH₂, —NHR₉, and —N(R₉)₂, substituted or unsubstituted heteroaryl, substituted or unsubstituted pyridinyl, substituted or unsubstituted pyridinyl, substituted or unsubstituted pyrimidinyl, substituted or unsubstituted pyrazolyl, substituted or unsubstituted triazolyl;
R₈ is selected from hydrogen, substituted or unsubstituted alkyl, substituted or unsubstituted aryl, substituted or unsubstituted heterocycloalkyl, substituted or unsubstituted heteroalkyl, and substituted or unsubstituted heteroaryl; and
R₉ is independently selected from hydrogen, —COR₈, —SO₂—R₈, substituted or unsubstituted alkyl, substituted or unsubstituted aryl, substituted or unsubstituted heteroalkyl, substituted or unsubstituted heteroalkenyl, substituted or unsubstituted heteroalkynyl, and substituted or unsubstituted heteroaryl.

2. The compound of claim 1, wherein Formula I is selected from: 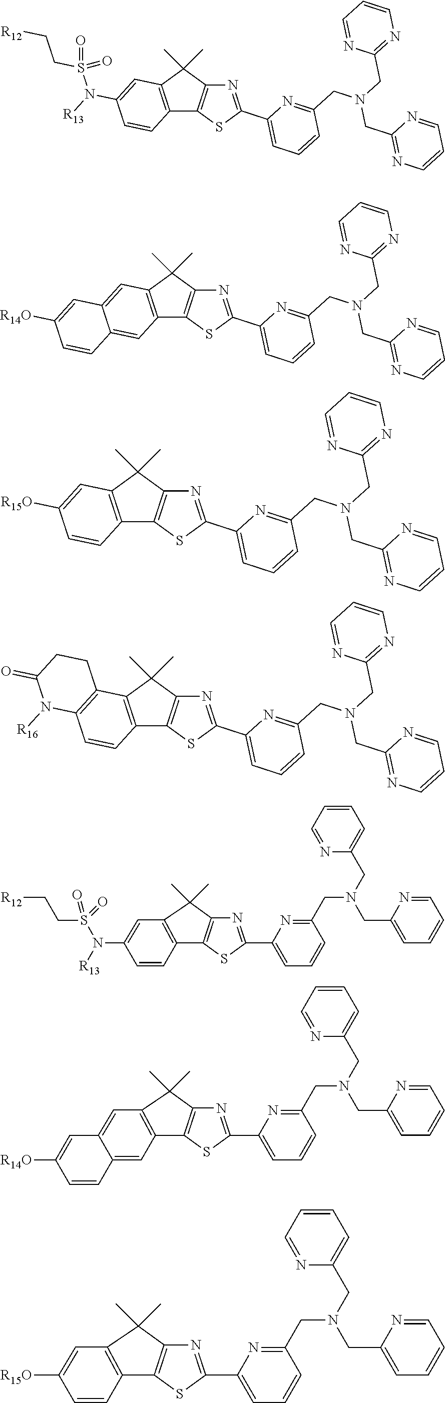 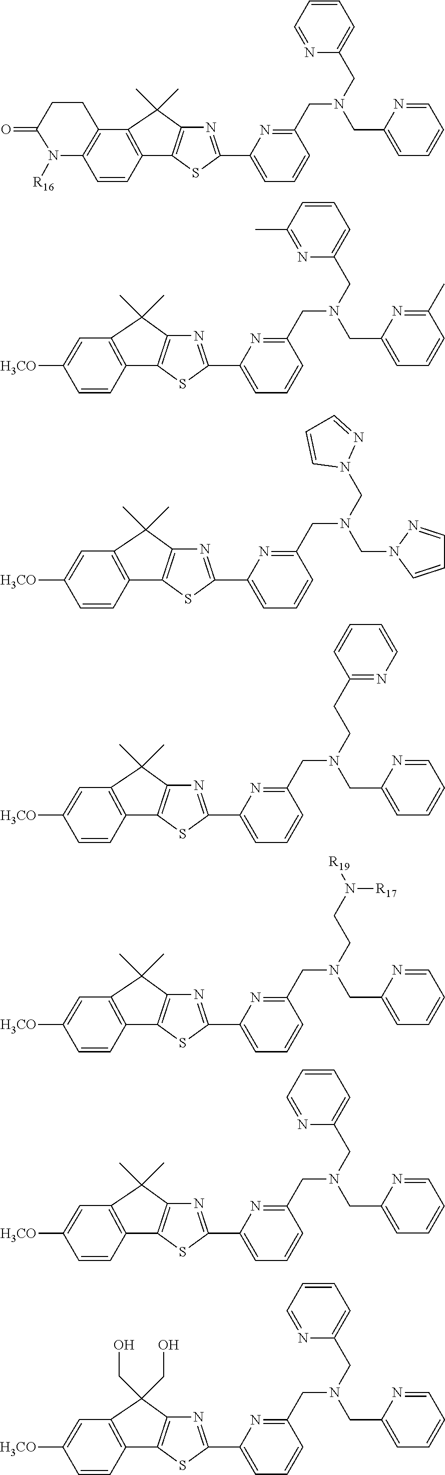 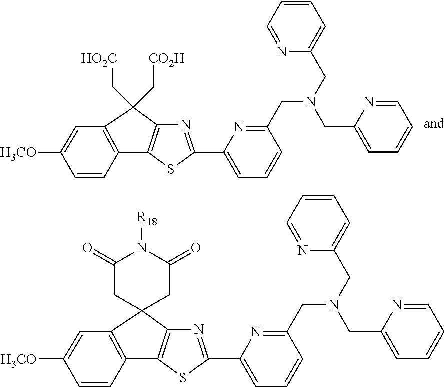
wherein
R₁₂, R₁₃, R₁₄, R₁₅, and R₁₆ are each independently selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl;
R₁₇ and R₁₉ are each independently selected from C₁₋₃-alkyl optionally substituted with —OR₈, —COOH, —COOR₈, —NHCOR₈, —NHR₉, and —N(R₉)₂, substituted or unsubstituted heteroaryl, substituted or unsubstituted pyridinyl, substituted or unsubstituted pyrimidinyl, substituted or unsubstituted pyrazolyl, substituted or unsubstituted triazolyl; and
R₁₈ is selected from a group consisting of hydrogen, substituted or unsubstituted heterocycloalkyl, substituted or unsubstituted heteroalkyl, and substituted or unsubstituted heteroaryl.

3. The compound of claim 1, wherein the compound comprises a conformationally locked core configured to be substituted with an electron donor moiety, wherein the substituted electron donor moiety is configured to fuse to a pyridine ring, wherein the pyridine ring is configured to act as an electron acceptor and metal ion binding moiety.

4. The compound of claim 3, wherein the compound allows for the modification of at least one of the following: electron donor moiety, bridging unit, chelator moiety, or a combination thereof.

5. The compound of claim 4, wherein the modification of the compound is configured to tune the spectral characteristics, attach functional groups for bioconjugation with proteins, immobilization on solid support, tailor an affinity for a specific application, or a combination thereof.

6. The compound of claim 1, wherein the compound is configured to exhibit a maximum absorption between about 400 nm and 500 nm for visible light or laser excitation.

7. The compound of claim 1, wherein the compound is configured to exhibit a maximum two-photon absorption cross section between about 750 nm and 950 nm for two-photon excitation.

8. The compound of claim 1, wherein the is configured to be utilized as an emission-ratiometric fluorescent probe for the detection of Zn(II) in at least one of the following processes: visible-light fluorescence microscopy, confocal laser fluorescence microscopy, light sheet fluorescence microscopy, two-photon excitation microscopy, fluorescence lifetime imaging microscopy (FLIM), flow cytometry, microplate fluorescence detection analysis, steady-state fluorescence spectroscopy, lifetime fluorescence spectroscopy, or a combination thereof.

9. The compound of claim 1, wherein the compound is a portion of a fluorescent complex, wherein the compound and zinc(II) comprising Formula I-Zn(II) is in a 1:1 stoichiometric ratio.

10. The compound of claim 9, wherein a maximum absorption wavelength of the Formula I-Zn(II) complex is from about 10 nm to about 60 nm greater than the maximum absorption wavelength of the compound of Formula I.

11. A compound according to the following formula: 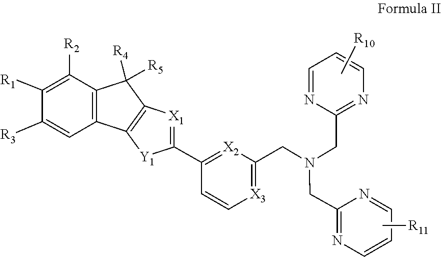
wherein:
Y₁ is selected from —O—, —S—, —N(R₄)—, and —C(R₄)(R₅)—;
X₁, X₂, and X₃ are independently selected from —CH— and —N—;
R₁ is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, substituted or unsubstituted heteroaryl, —OCH₃, —OCH₂COOR₉, —OCH₂CH₂COOR₉, —OCH₂CH₂CH₂SO₃R₉, —NHR₉, and —N(R₉)₂;
R₂ is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl; or in the alternative, R₁ and R₂ join together to form a donor selected from substituted or unsubstituted heterocycloalkyl, substituted or unsubstituted heterocycloalkenyl, and substituted or unsubstituted heteroaryl;
R₃ is selected from hydrogen, substituted or unsubstituted alkyl, substituted or unsubstituted alkenyl, substituted or unsubstituted alkynyl, substituted or unsubstituted cycloalkyl, substituted or unsubstituted cycloalkenyl, substituted or unsubstituted aryl, substituted or unsubstituted heteroalkyl, substituted or unsubstituted heteroalkenyl, substituted or unsubstituted heteroalkynyl, substituted or unsubstituted heterocycloalkyl, substituted or unsubstituted heterocycloalkenyl, and substituted or unsubstituted heteroaryl; or in the alternative, R₁ and R₃ join together to form a donor selected from substituted or unsubstituted heterocycloalkyl, substituted or unsubstituted heterocycloalkenyl, and substituted or unsubstituted heteroaryl;
R₄ and R₅ are each independently selected from hydrogen, substituted or unsubstituted heterocycloalkyl, substituted or unsubstituted heteroalkyl, and substituted or unsubstituted heteroaryl;
R₈ is selected from hydrogen, substituted or unsubstituted alkyl, substituted or unsubstituted aryl, substituted or unsubstituted heterocycloalkyl, substituted or unsubstituted heteroalkyl, and substituted or unsubstituted heteroaryl;
R₉ is independently selected from hydrogen, —COR₈, —SO₂—R₈, substituted or unsubstituted alkyl, substituted or unsubstituted aryl, substituted or unsubstituted heteroalkyl, substituted or unsubstituted heteroalkenyl, substituted or unsubstituted heteroalkynyl, and substituted or unsubstituted heteroaryl; and
R₁₀ and R₁₁ are each independently selected from —H, —CH₃, —OR₈, —CF₃, —F, —Cl, —CN, —COOR₈, and —SO₃R₈.

12. The compound of claim 11, wherein the compound is configured to exhibit a maximum absorption between about 380 nm and 500 nm for visible light or laser excitation, wherein the compound is configured to exhibit a maximum two-photon absorption cross section between about 720 nm and 950 nm for two-photon excitation.

13. The compound of claim 11, wherein the compound is configured to be utilized as an emission-ratiometric fluorescent probe for the detection of Zn(II) in at least one of the following processes: visible-light fluorescence microscopy, confocal laser fluorescence microscopy, light sheet fluorescence microscopy, two-photon excitation microscopy, fluorescence lifetime imaging microscopy (FLIM), flow cytometry, microplate fluorescence detection analysis, steady-state fluorescence spectroscopy, lifetime fluorescence spectroscopy, or a combination thereof.

14. The compound of claim 11, wherein the compound is configured to be a portion of a fluorescent complex, wherein the compound and zinc(II) comprising Formula II-Zn(II) is in a 1:1 stoichiometric ratio.

15. The compound of claim 14, wherein a maximum absorption wavelength of the Formula II-Zn(II) complex is from about 10 nm to about 60 nm greater than the maximum absorption wavelength of the compound of Formula II.

16. A method of detecting zinc in a sample, the method comprising:
treating the sample with a compound according to Formula I or Formula II; 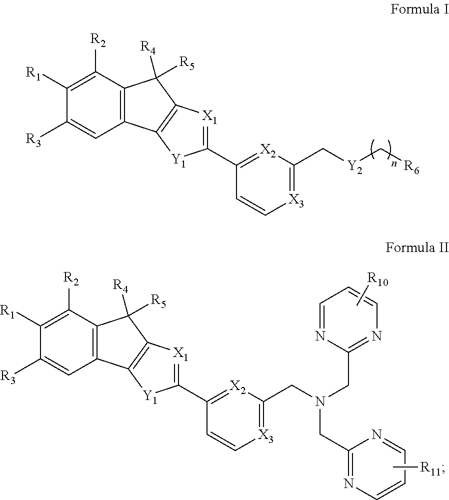
detecting an integrated fluorescence emission intensity over two different wavelength ranges, wherein the first wavelength range is dominated by fluorescence emission of the compound and the second wavelength range is dominated by fluorescence emission of the zinc(II) complex of the compound; and
comparing a first integrated emission intensity wavelength range to a second integrated emission intensity wavelength range.

17. The method of claim 16, wherein the compound is configured to exhibit a fluorescence emission maximum between about 420 to 650 nm, wherein an emission maximum of the Zn(II) complex of a compound is from about 10 nm to about 60 n.

18. The method of claim 17, wherein the method is configured to utilize two-photon excitation, wherein causing the compound to shift the emission maximum by about 10 nm to about 60 nm upon formation of a complex with Zn(II).

19. The method of claim 16, wherein the method further comprises:
detecting Zn(II) using ratiometric fluorescence analysis of the compound emission over two different wavelength ranges, by comparing the integrated emission intensity over first wavelength to the integrated emission intensity over the second wavelength range.

20. The method of claim 16, wherein the method further comprises:
determining a fractional saturation of the compound with Zn(II) by comparing the first integrated emission intensity wavelength range to the second integrated emission intensity wavelength range, wherein the fractional saturation of the compound with Zn(II) determines the concentration and/or activity of Zn(II) in the treated sample.
